(19) United States
(12) Patent Application Publication
(10) Pub. No.: US 2007/0299115 A1
(43) Pub. Date: Dec. 27, 2007

(54) (Hetero)Cyclyl(Thio) Carboxylic Acid Anilides For Controlling Pathogenic Fungi

(75) Inventors: Markus Gewehr, Kastellaun (DE); Bernd Muller, Frankenthal (DE); Thomas Grote, Wachenheim (DE); Wassilios Grammenos, Ludwigshafen (DE); Anja Shwogler, Mannheim (DE); Joachim Rheinheimer, Ludwigshafen (DE); Carsten Blettner, Hong Kong (CN); Peter Schafer, Ottersheim (DE); Frank Schieweck, Bad Durkheim (DE); Oliver Wagner, Neustadt (DE); Jan Rether, Kaiserslautern (DE); Siegfried Strathmann, Limburgerhof (DE); Reinhard Stierl, Kaohsiung Country (TW); Maria Scherer, Landau (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 11/661,679

(22) PCT Filed: Sep. 5, 2005
(86) PCT No.: PCT/EP05/09529

§ 371 Date: May 11, 2007

(30) Foreign Application Priority Data
Sep. 6, 2004 (DE) 10 2004 043 047.0

(51) Int. Cl. A01N 37/48; A01N 43/40; A01N 43/56; A01N 43/78; A01P 3/00; C07C 233/00; C07D 213/82; C07D 231/14; C07D 277/56

(52) U.S. Cl. 514/354; 514/365; 514/406; 514/563; 546/337; 548/200; 548/374.1; 564/161

(57) ABSTRACT

The invention relates to (hetero)cyclyl(thio)carboxylic acid anilides of general formula (I) and to salts of said anilides that can be used for agricultural purposes for controlling pathogenic fungi. In said formula, the variables are defined as follows: A represents a phenyl or at least a monounsaturated 5- or 6-membered heterocycle comprising 1, 2 or 3 heteroatoms, selected from N, O, S, S(═O) and S(═O)₂ as ring members, whereby phenyl and the monounsaturated 5- or 6-membered heterocycle can be unsubstituted or can be substituted according to the description; B represents a group of general formula (II), in which the variables R³, R⁴, R⁵ and the index m are defined as cited in the claims and the description; Y represents oxygen or sulphur, R¹ represents H, OH, alkyl, cycloalkyl, alkoxy, haloalkyl, halocycloalkyl or haloalkoxy; R² represents halogen, nitro, CN, alkyl, cycloalkyl, alkenyl, alkynyl, alkoxy, haloalkyl, halocycloalkyl, haloalkenyl, haloalkynyl or haloalkoxy; and n represents 0, 1, 2, 3 or 4; and salts that can be used for agricultural purposes. The invention also relates to the use of the (hetero)cyclyl(thio)carboxylic acid anilides of general formula (I), to a method for controlling pathogenic fungi and to a crop protection agent containing at least one compound of general formula (I) and/or an agriculturally compatible salt of said compound.

The present invention relates to (hetero)cyclic carboxanilides having an oxime ether function and their use for controlling harmful fungi.

WO 02/08197 describes fungicidally active hetarylcarboxanilides having a phenyl group in the 2-position on the phenyl ring, which phenyl group carries an oxime ether group. 1,3-Dimethyl-5-fluoropyrazole-4-carboxanilides of a similar structure are known from WO 02/08197.

WO 98/03500 describes hetarylcarboxanilides which may, inter alia, have a phenoxy group on the phenyl ring.

WO 95/01339 discloses 4-pyridinecarboxanilides which carry a phenoxy substituent in the 2-position of the anilide ring.

However, the (heteroaryl)carboxanilides described in the prior art are, in particular at low application rates, not entirely satisfactory.

Accordingly, it is an object of the present invention to provide fungicidally active compounds which overcome the disadvantages of the compounds known from the prior art and, in particular, have improved action at low application rates. Moreover, these compounds should have good compatibility with useful plants and, if possible, cause little, if any, harm to useful animals.

This object is achieved by the (hetero)cyclyl(thio)carboxanilides of the formula I described below and by their agriculturally acceptable salts.

Accordingly, the present invention relates to (hetero)cyclyl(thio)carboxanilides of the formula I, 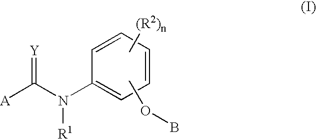
in which the variables are as defined below:
A is phenyl or an at least monounsaturated 5- or 6-membered heterocycle having 1, 2 or 3 heteroatoms selected from the group consisting of N, O, S, S(═O) and S(═O)₂ as ring members, where phenyl and the at least monounsaturated 5- or 6-membered heterocycle may be unsubstituted or may carry 1, 2 or 3 radicals R^{a}, where
   R^{a} is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl, C₁-C₄-haloalkoxy or phenyl, where phenyl may be unsubstituted or carries one, two or three radicals R^{b} selected from the group consisting of halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy;
B is a radical of the formula 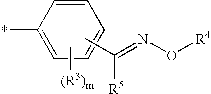
Y is oxygen or sulfur;
R¹ is H, OH, C₁-C₄-alkyl, C₃-C₆-cycloalkyl, C₁-C₄-alkoxy, C₁-C₄-haloalkyl, C₃-C₆-halocycloalkyl or C₁-C₄-haloalkoxy;
R², R³ independently of one another are halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl or C₁-C₄-haloalkoxy;
R⁴ is hydrogen, C₁-C₈-alkyl, C₃-C₆-cycloalkyl, C₂-C₈-alkenyl, C₂-C₈-alkynyl, C₁-C₈-haloalkyl, C₃-C₆-halocycloalkyl, C₂-C₈-haloalkenyl, C₂-C₈-haloalkynyl, phenyl, naphthyl, phenyl-C₁-C₄-alkyl, naphthyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl or phenyl-C₂-C₄-haloalkynyl, where phenyl and naphthyl in the 9 lastmentioned groups may be unsubstituted or may carry 1, 2 or 3 substituents selected from the group consisting of R^{b} and R⁶, where
R⁶ is —(CR⁷)═NOR⁸ in which
   R⁷ is hydrogen, C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, benzyl; where phenyl and the phenyl group in benzyl may be unsubstituted or may carry one, two or three radicals R^{b}; and
   R⁸ is C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl, phenyl-C₂-C₄-haloalkynyl, where phenyl in the 7 lastmentioned radicals may be unsubstituted or may carry one, two or three radicals R^{b};
R⁵ is hydrogen, C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl or phenyl-C₂-C₄-haloalkynyl, where phenyl in the 7 lastmentioned radicals may be unsubstituted or may carry one, two or three radicals R^{b};
n is 0, 1, 2, 3 or 4; and
m is 0, 1, 2 or 3;
   and their agriculturally useful salts, with the exception of compounds of the formula I in which A is 4-pyridyl.

Moreover, the present invention relates to the use of the (hetero)cyclyl(thio)carbox-anilides of the formula I and their agriculturally acceptable salts as fungicides, and to crop protection compositions comprising these compounds.

Furthermore, the present invention relates to a method for controlling phytopathogenic fungi (harmful fungi), which method comprises treating the harmful fungi, their habitat or the plants, areas, materials or spaces to be kept free from them with a fungicidally effective amount of a (hetero)cyclylcarboxamide of the formula I and/or an agriculturally useful salt of I.

Depending on the substitution pattern, the compounds of the formula I may have one or more centers of chirality, in which case they are present as enantiomer or diastereomer mixtures. The invention provides both the pure enantiomers or diastereomers and also their mixtures. Suitable compounds of the formula I also include all possible stereoisomers (cis/trans isomers) and mixtures thereof.

Suitable agriculturally useful salts are especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and also the anions of C₁-C₄-alkanoic acids, preferably formate, acetate, propionate and butyrate. They can be formed by reacting I with an acid of the corresponding anion, preferably of hydrochloric acid, hydrobromic acid, sulfuric acid, phosphoric acid or nitric acid.

In the definitions of the variables given in the formulae above, collective terms are used which are generally representative of the substituents in question. The term Cₙ-Cₘ denotes the number of carbon atoms possible in each case in the respective substituent or substituent moiety. All carbon chains, i.e. all alkyl, haloalkyl, phenylalkyl, alkenyl, haloalkenyl, phenylalkenyl, alkynyl, haloalkynyl and phenylalkynyl moieties may be straight-chain or branched. Halogenated substituents preferably carry one to five identical or different halogen atoms. The term “halogen” denotes in each case fluorine, chlorine, bromine or iodine.

Examples of other meanings are:
C₁-C₄-alkyl: CH₃, C₂H₅, CH₂—C₂H₅, CH(CH₃)₂, n-butyl, CH(CH₃)—C₂H₅, CH₂—CH(CH₃)₂ or C(CH₃)₃;
C₁-C₄-haloalkyl: a C₁-C₄-alkyl radical as mentioned above which is partially or fully substituted by fluorine, chlorine, bromine and/or iodine, i.e., for example, CH₂F, CHF₂, CF₃, CH₂Cl, CH(Cl)₂, C(Cl)₃, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, C₂F₅, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl;
C₁-C₈-alkyl: a C₁-C₄-alkyl radical as mentioned above or, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably CH₃, C₂H₅, CH₂—C₂H₅, CH(CH₃)₂, n-butyl, C(CH₃)₃, n-pentyl, n-hexyl, n-heptyl or n-octyl;
C₁-C₈-haloalkyl: a C₁-C₈-alkyl radical as mentioned above which is partially or fully substituted by fluorine, chlorine, bromine and/or iodine, i.e., for example, one of the radicals mentioned under C₁-C₄-haloalkyl or 5-fluoro-1-pentyl, 5-chloro-1-pentyl, 5-bromo-1-pentyl, 5-iodo-1-pentyl, 5,5,5-trichloro-1-pentyl, undecafluoropentyl, 6-fluoro-1-hexyl, 6-chloro-1-hexyl, 6-bromo-1-hexyl, 6-iodo-1-hexyl, 6,6,6-trichloro-1-hexyl or dodecafluorohexyl;
C₂-C₄-alkenyl: unsaturated straight-chain or branched hydrocarbon radicals having 2 to 4 carbon atoms and a double bond in any position, for example ethenyl, 1-propenyl, 2-propenyl, 1-methylethenyl, 1-buten-1-yl, 1-buten-2-yl, 1-buten-3-yl, 2-buten-1-yl, 1-methylprop-1-en-1-yl, 2-methylprop-1-en-1-yl, 1-methylprop-2-en-1-yl, 2-methylprop-2-en-1-yl;
C₂-C₆-alkenyl: C₂-C₄-alkenyl as mentioned above and also, for example, n-penten-1-yl, n-penten-2-yl, n-penten-3-yl, n-penten-4-yl, 1-methylbut-1-en-1-yl, 2-methylbut-1-en-1-yl, 3-methylbut-1-en-1-yl, 1-methylbut-2-en-1-yl, 2-methylbut-2-en-1-yl, 3-methylbut-2-en-1-yl, 1-methylbut-3-en-1-yl, 2-methylbut-3-en-1-yl, 3-methylbut-3-en-1-yl, 1,1-dimethylprop-2-en-1-yl, 1,2-dimethylprop-1-en-1-yl, 1,2-dimethylprop-2-en-1-yl, 1-ethylprop-1-en-2-yl, 1-ethylprop-2-en-1-yl, n-hex-1-en-1-yl, n-hex-2-en-1-yl, n-hex-3-en-1-yl, n-hex-4-en-1-yl, n-hex-5-en-1-yl, 1-methylpent-1-en-1-yl, 2-methylpent-1-en-1-yl, 3-methylpent-1-en-1-yl, 4-methylpent-1-en-1-yl, 1-methylpent-2-en-1-yl, 2-methylpent-2-en-1-yl, 3-methylpent-2-en-1-yl, 4-methylpent-2-en-1-yl, 1-methylpent-3-en-1-yl, 2-methylpent-3-en-1-yl, 3-methylpent-3-en-1-yl, 4-methylpent-3-en-1-yl, 1-methylpent-4-en-1-yl, 2-methylpent-4-en-1-yl, 3-methylpent-4-en-1-yl, 4-methylpent-4-en-1-yl, 1,1-dimethylbut-2-en-1-yl, 1,1-dimethylbut-3-en-1-yl, 1,2-dimethylbut-1-en-1-yl, 1,2-dimethylbut-2-en-1-yl, 1,2-dimethylbut-3-en-1-yl, 1,2-dimethylbut-1-en-1-yl, 1,3-dimethyl-but-2-en-1-yl, 1,3-dimethylbut-3-en-1-yl, 2,2-dimethylbut-3-en-1-yl, 2,3-dimethylbut-1-en-1-yl, 2,3-dimethylbut-2-en-1-yl, 2,3-dimethylbut-3-en-1-yl, 3,3-dimethylbut-1-en-1-yl, 3,3-dimethylbut-2-en-1-yl, 1-ethylbut-1-en-1-yl, 1-ethylbut-2-en-1-yl, 1-ethylbut-3-en-1-yl, 2-ethylbut-1-en-1-yl, 2-ethylbut-2-en-1-yl, 2-ethylbut-3-en-1-yl, 1,1,2-trimethylprop-2-en-1-yl, 1-ethyl-1-methylprop-2-en-1-yl, 1-ethyl-2-methylprop-1-en-1-yl or 1-ethyl-2-methylprop-2-en-1-yl;
C₂-C₄-haloalkenyl: unsaturated straight-chain or branched hydrocarbon radicals having 2 to 4 carbon atoms and a double bond in any position (as mentioned above), where in these groups some or all of the hydrogen atoms are replaced by halogen atoms as mentioned above, in particular by fluorine, chlorine and bromine, i.e., for example, 2-chloroallyl, 3-chloroallyl, 2,3-dichloroallyl, 3,3-dichloroallyl, 2,3,3-trichloroallyl, 2,3-dichlorobut-2-enyl, 2-bromoallyl, 3-bromoallyl, 2,3-dibromoallyl, 3,3-dibromoallyl, 2,3,3-tribromoallyl or 2,3-dibromobut-2-enyl;
C₂-C₆-haloalkenyl: C₂-C₆-alkenyl as mentioned above which is partially or fully substituted by fluorine, chlorine, bromine and/or iodine, for example the radicals mentioned under C₂-C₄-haloalkenyl;
C₂-C₄-alkynyl: straight-chain or branched hydrocarbon groups having 2 to 4 carbon atoms and a triple bond in any position, for example ethynyl, 1-propynyl, 2-propynyl (=propargyl), 1-butynyl, 2-butynyl, 3-butynyl and 1-methyl-2-propynyl;
C₂-C₆-alkynyl: straight-chain or branched hydrocarbon groups having 2 to 6 carbon atoms and a triple bond in any position, for example ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl and 4-methylpent-2-yn-5-yl;
C₂-C₄-haloalkynyl: unsaturated straight-chain or branched hydrocarbon radicals having 2 to 4 carbon atoms and a triple bond in any position (as mentioned above), where in these groups some or all of the hydrogen atoms may be replaced by halogen atoms as mentioned above, in particular by fluorine, chlorine and bromine, i.e., for example, 1,1-difluoroprop-2-yn-1-yl, 4-fluorobut-2-yn-1-yl, 4-chlorobut-2-yn-1-yl or 1,1-difluorobut-2-yn-1-yl;
C₂-C₆-haloalkynyl: C₂-C₆-alkynyl as mentioned above which is partially or fully substituted by fluorine, chlorine, bromine and/or iodine, for example the radicals mentioned under C₂-C₄-haloalkynyl;
C₁-C₄-alkoxy: OCH₃, OC₂H₅, OCH₂—C₂H₅, OCH(CH₃)₂, n-butoxy, OCH(CH₃)—C₂H₅, OCH₂—CH(CH₃)₂ or OC(CH₃)₃;
C₁-C₄-haloalkoxy: a C₁-C₄-alkoxy radical as mentioned above which is partially or fully substituted by fluorine, chlorine, bromine and/or iodine, i.e., for example, OCH₂F, OCHF₂, OCF₃, OCH₂Cl, OCH(Cl)₂, OC(Cl)₃, chlorofluoromethoxy, dichorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy, preferably OCHF₂, OCF₃, dichlorofluoromethoxy, chlorodifluoromethoxy or 2,2,2-trifluoroethoxy;
C₃-C₆-cycloalkyl: cyclopropyl, cyclobutyl, cyclopentyl or cyclohexyl;
C₃-C₆-cycloalkyl which is optionally mono- or polysubstituted by halogen: a C₃-C₆-cycloalkyl radical as mentioned above which is unsubstituted or partially or fully substituted by fluorine, chlorine, bromine and/or iodine, i.e., for example, 1-chlorocyclopropyl, 1-fluorocyclopropyl, 2-chlorocyclopropyl, 2-fluorocyclopropyl, 4-chlorocyclohexyl, 4-bromocyclohexyl;
phenyl-C₁-C₄-alkyl: C₁-C₄-alkyl which is substituted by phenyl, for example benzyl, 1- or 2-phenylethyl, 1-, 2- or 3-phenylpropyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 lastmentioned groups may be substituted by halogen;
naphthyl-C₁-C₄-alkyl: C₁-C₄-alkyl which carries an α- or β-naphthyl radical, for example α- or β-naphthylmethyl, 1- or 2-(α- or β-naphthyl)ethyl, 1-, 2- or 3-(α- or β-naphthyl)propyl, where the naphthyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 lastmentioned groups may be substituted by halogen;
phenyl-C₁-C₄-haloalkyl: C₁-C₄-haloalkyl which is substituted by phenyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 lastmentioned groups may be substituted by halogen;
phenyl-C₂-C₄-alkenyl: C₂-C₄-alkenyl which is substituted by phenyl, for example 1- or 2-phenylethenyl, 1-phenylprop-2-en-1-yl, 3-phenyl-1-propen-1-yl, 3-phenyl-2-propen-1-yl, 4-phenyl-1-buten-1-yl or 4-phenyl-2-buten-1-yl; where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 lastmentioned groups may be substituted by halogen;
phenyl-C₂-C₄-haloalkenyl: C₂-C₄-haloalkenyl which is substituted by phenyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 lastmentioned groups may be substituted by halogen;
phenyl-C₂-C₄-alkynyl: C₂-C₄-alkynyl which is substituted by phenyl, for example 1-phenyl-2-propyn-1-yl, 3-phenyl-1-propyn-1-yl, 3-phenyl-2-propyn-1-yl, 4-phenyl-1-butyn-1-yl or 4-phenyl-2-butyn-1-yl; where the phenyl moiety of phenyl-C₂-C₄-alkynyl may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 lastmentioned groups may be substituted by halogen;
phenyl-C₂-C₄-haloalkynyl: C₂-C₄-haloalkynyl which is substituted by phenyl, where the phenyl moiety may be unsubstituted or may carry 1, 2 or 3 radicals R^{b}, where R^{b} is selected from the group consisting of halogen, nitro, CN, C₁-C₄-alkyl, C₃-C₆-cycloalkyl, C₂-C₄-alkenyl, C₂-C₄-alkynyl and C₁-C₄-alkoxy, where the 5 lastmentioned groups may be substituted by halogen;
an at least monounsaturated heterocycle having 5 or 6 ring members: a monocyclic heterocycle which has one, two or three ring members selected from the group consisting of O, S, S(═O), S(═O)₂ and N and is at least monounsaturated or fully unsaturated, i.e. aromatic. Examples of these are furyl, such as 2-furyl and 3-furyl, thienyl, such as 2-thienyl and 3-thienyl, pyrrolyl, such as 2-pyrrolyl and 3-pyrrolyl, isoxazolyl, such as 3-isoxazolyl, 4-isoxazolyl and 5-isoxazolyl, isothiazolyl, such as 3-isothiazolyl, 4-isothiazolyl and 5-isothiazolyl, pyrazolyl, such as 3-pyrazolyl, 4-pyrazolyl and 5-pyrazolyl, oxazolyl, such as 2-oxazolyl, 4-oxazolyl and 5-oxazolyl, thiazolyl, such as 2-thiazolyl, 4-thiazolyl and 5-thiazolyl, imidazolyl, such as 2-imidazolyl and 4-imidazolyl, oxadiazolyl, such as 1,2,4-oxadiazol-3-yl, 1,2,4-oxadiazol-5-yl and 1,3,4-oxadiazol-2-yl, thiadiazolyl, such as 1,2,4-thiadiazol-3-yl, 1,2,4-thiadiazol-5-yl and 1,3,4-thiadiazol-2-yl, triazolyl, such as 1,2,4-triazol-1-yl, 1,2,4-triazol-3-yl and 1,2,4-triazol-4-yl, pyridinyl, such as 2-pyridinyl, 3-pyridinyl and 4-pyridinyl, pyridazinyl, such as 3-pyridazinyl and 4-pyridazinyl, pyrimidinyl, such as 2-pyrimidinyl, 4-pyrimidinyl and 5-pyrimidinyl, 2-pyrazinyl, 1,3,5-triazin-2-yl and 1,2,4-triazin-3-yl, 1,2-dihydrofuran-2-yl, 1,2-dihydrofuran-3-yl, 1,2-dihydrothiophen-2-yl, 1,2-dihydrothiophen-3-yl, 2,3-dihydropyran-4-yl, 2,3-dihydropyran-5-yl, 2,3-dihydropyran-6-yl, 5,6-dihydro-4H-pyran-3-yl, 2,3-dihydrothiopyran-4-yl, 2,3-dihydrothiopyran-5-yl, 2,3-dihydrothiopyran-6-yl, 5,6-dihydro-4H-thiopyran-3-yl, 5,6-dihydro-[1,4]dioxin-2-yl, 5,6-dihydro-[1,4]dithiin-2-yl or 5,6-dihydro-[1,4]oxathiin-3-yl, in particular pyridyl, thiazolyl and pyrazolyl.

With a view to the fungicidal activity of the compounds I according to the invention, preference is given to those compounds of the formula I in which A is a cyclic radical A-1 to A-6: 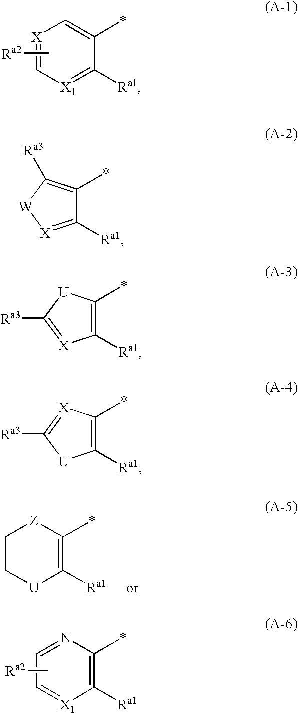
in which * denotes the point of attachment to C(═Y) and the variables are as defined below:
X, X₁ in each case independently of one another are N or CR^{c}, where R^{c} is H or has the meanings given for R^{b}. R^{c} is in particular hydrogen;
W is S or N—R^{a4} in which R^{a4} is hydrogen, C₁-C₄-alkyl, C₁-C₄-haloalkyl, C₁-C₄-alkoxy, C₁-C₄-haloalkyl or phenyl which may be unsubstituted or may carry 1, 2 or 3 radicals R^{b}, where R^{a4} is in particular hydrogen, C₁-C₄-alkyl or C₁-C₄-haloalkyl;
U is oxygen or sulfur;
Z is S, S(═O), S(═O)₂ or CH₂, particularly preferably S or CH₂;
R^{a1} is hydrogen, C₁-C₄-alkyl, C₁-C₄-alkoxy, C₁-C₄-haloalkyl, C₁-C₄-haloalkoxy or halogen, particularly preferably hydrogen, halogen, C₁-C₂-alkyl, C₁-C₂-alkoxy, C₁-C₂-fluoroalkoxy or C₁-C₂-fluoroalkyl;
R^{a2} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 lastmentioned groups may be substituted by halogen; and
R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 lastmentioned groups may be substituted by halogen, particularly preferably hydrogen, fluorine, chlorine or C₁-C₄-alkyl.

In the radicals of the formulae A-1, A-2, A-3, A-4, A-5 and A-6, the variables R^{a1}, R^{a2} and R^{a3} have in particular the following meanings:
R^{a1} is hydrogen, halogen, in particular fluorine or chlorine, C₁-C₄-alkyl or C₁-C₄-haloalkyl, particularly preferably halogen, trifluoromethyl or methyl;
R^{a2} is hydrogen; and
R^{a3} is halogen, in particular fluorine or chlorine, or methyl.

In the formula A-2, W is preferably a group N—R^{a4} in which R^{a4} has the meanings given above and in particular the meanings given as being preferred.

If X in the formulae A-1, A-2, A-3 or A-4 is a group C—R^{c}, R^{c} is preferably hydrogen.

X in the formulae A-2, A-3 and A-4 is in particular N. In formula A-1, X is in particular CH.

In the formula A-1, X₁ is in particular N. In a preferred embodiment, A is A-6 in which X₁ is N. In a further preferred embodiment, A is A-6 in which X₁ is C—R^{c} and in particular C—H.

Examples of radicals A-1 are in particular: 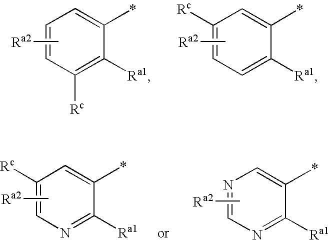
in which *, R^{a1}, R^{a2} and R^{c} have the meanings given above and in particular the preferred meanings.

Examples of radicals A-2 are in particular: 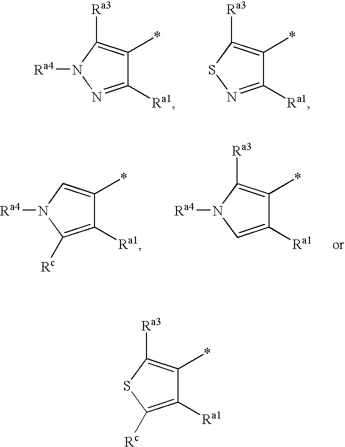
in which *, R^{a1}, R^{a3}, R^{a4} and R^{c} have the meanings given above and in particular the preferred meanings.

Examples of radicals A-3 are in particular: 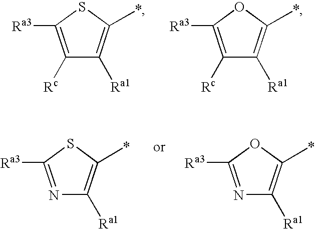
in which *, R^{a1}, R^{a3} and R^{c} have the meanings given above and in particular the preferred meanings.

Examples of radicals A-4 are in particular: 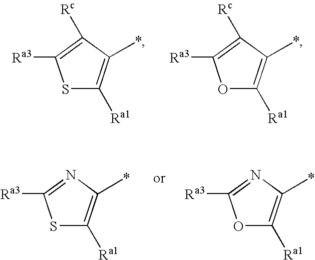
in which *, R^{a1}, R^{a3} and R^{c} have the meanings given above and in particular the preferred meanings.

Examples of A-5 are in particular: 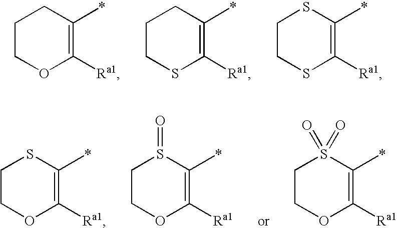
in which * and R^{a1} have the meanings given above and in particular the preferred meanings.

Examples of A-6 are in particular: 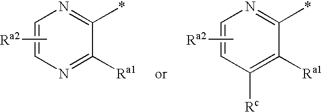
in which *, R^{a1}, R^{a2} and R^{c} have the meanings given above and in particular the preferred meanings.

Examples of radicals A are: 2-chlorophenyl, 2-trifluoromethylphenyl, 2-difluoromethylphenyl, 2-methylphenyl, 2-chloropyridin-3-yl, 2-trifluoromethylpyridin-3-yl, 2-difluoromethylpyridin-3-yl, 2-methylpyridin-3-yl, 4-methylpyrimidin-5-yl, 4-trifluoromethylpyrimidin-5-yl, 4-difluoromethylpyrimidin-5-yl, 1-methyl-3-trifluoromethylpyrazol-4-yl, 1-methyl-3-difluoromethylpyrazol-4-yl, 1,3-dimethylpyrazol-4-yl, 1-methyl-3-trifluoromethyl-5-fluoropyrazol-4-yl, 1-methyl-3-difluoromethyl-5-fluoropyrazol-4-yl, 1-methyl-3-trifluoromethyl-5-chloropyrazol-4-yl, 1-methyl-3-trifluoromethylpyrrol-4-yl, 1-methyl-3-difluoromethylpyrrol-4-yl, 2-methyl-4-trifluoromethylthiazol-5-yl, 2-methyl-4-difluoromethylthiazol-5-yl, 2,4-dimethylthiazol-5-yl, 2-methyl-5-trifluoromethylthiazol-4-yl, 2-methyl-5-difluoromethylthiazol-4-yl, 2,5-dimethylthiazol-4-yl, 2-methyl-4-trifluoromethyloxazol-5-yl, 2-methyl-4-difluoromethyloxazol-5-yl, 2,4-dimethyloxazol-5-yl, 2-trifluoromethylthiophen-3-yl, 5-methyl-2-trifluoromethylthiophen-3-yl, 2-methylthiophen-3-yl, 2,5-dimethylthiophen-3-yl, 3-trifluoromethylthiophen-2-yl, 3-methylthiophen-2-yl, 3,5-dimethylthiophen-2-yl, 5-methyl-3-trifluoromethylthiophen-2-yl, 2-trifluoromethylfuran-3-yl, 5-methyl-2-trifluoromethylfuran-3-yl, 2-methylfuran-3-yl, 2,5-dimethylfuran-3-yl, 2-methyl-5,6-dihydro-[1,4]oxathiin-3-yl, 2-methyl-5,6-dihydro-4H-thiopyran-3-yl.

With particular preference, A is a radical A-1a, A-2a or A-3a, 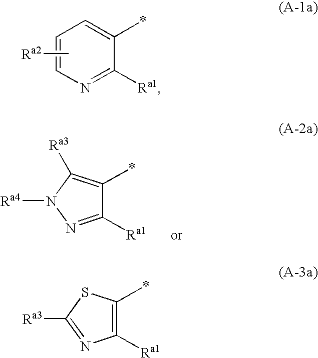
in which *, R^{a1}, R^{a2}, R^{a3} and R^{a4} have the meanings given above and in particular the preferred meanings.

Preference is given to radicals A-1a where R^{a1} is hydrogen, halogen, C₁-C₂-alkyl, C₁-C₂-alkoxy, C₁-C₂-fluoroalkoxy or C₁-C₂-fluoroalkyl; in particular hydrogen, chlorine, bromine, fluorine, methyl, ethyl, methoxy, trifluoromethyl, difluoromethyl, trifluoromethoxy or difluoromethoxy, very particularly preferably fluorine, bromine, chlorine, methyl or trifluoromethyl, and especially chlorine; where R^{a2} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 lastmentioned groups may be substituted by halogen, especially hydrogen.

Preference is given to radicals A-2a where R^{a1} is hydrogen, halogen, C₁-C₂-alkyl, C₁-C₂-alkoxy, C₁-C₂-fluoroalkoxy or C₁-C₂-fluoroalkyl, in particular hydrogen, chlorine, bromine, fluorine, methyl, ethyl, methoxy, trifluoromethyl, difluoromethyl, trifluoromethoxy or difluoromethoxy, very particularly preferably fluorine, bromine, chlorine, methyl or trifluoromethyl, especially trifluoromethyl; R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 lastmentioned groups may be substituted by halogen, preferably hydrogen, halogen and C₁-C₄-alkyl, in particular halogen, hydrogen; and especially hydrogen; and R^{a4} is hydrogen, C₁-C₄-alkyl, C₁-C₄-haloalkyl, C₁-C₄-alkoxy, C₁-C₄-haloalkyl or phenyl which may be unsubstituted or may carry 1, 2 or 3 radicals R^{b}, preferably hydrogen, C₁-C₄-alkyl or C₁-C₄-haloalkyl, especially methyl;

Preference is given to radicals A-3a where R^{a1} is hydrogen, halogen, C₁-C₂-alkyl, C₁-C₂-alkoxy, C₁-C₂-fluoroalkoxy or C₁-C₂-fluoroalkyl, in particular hydrogen, chlorine, bromine, fluorine, methyl, ethyl, methoxy, trifluoromethyl, difluoromethyl, trifluoromethoxy or difluoromethoxy, very particularly preferably fluorine, bromine, chlorine, methyl or trifluoromethyl, especially trifluoromethyl; R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 lastmentioned groups may be substituted by halogen, preferably hydrogen, halogen or C₁-C₄-alkyl, in particular hydrogen, methyl and especially methyl.

With particular preference, A is selected from the group consisting of: A-1a where R^{a1}=halogen, especially chlorine, and R^{a2}=hydrogen; A-2a where R^{a1}═C₁-C₂-fluoroalkyl, especially trifluoromethyl, R^{a3}=hydrogen and R^{a4}═C₁-C₄-alkyl, especially methyl; and A-3a where R^{a1}═C₁-C₂-fluoroalkyl, especially trifluoromethyl, and R^{a3}═C₁-C₄-alkyl, especially methyl.

Among the (hetero)cyclylcarboxamides according to the invention, preference is given to those compounds of the formula I in which the group O—B is attached in the ortho-position to the group N—R₁, i.e. compounds of the formula I′ given below 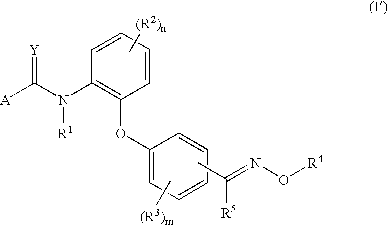
where the variables n, m, A, Y, R¹, R², R³, R⁴ and R⁵ have the meanings given above and in particular the meanings given here and below as being preferred or particularly preferred.

Among the (hetero)cyclylcarboxamides according to the invention, preference is furthermore given to those compounds of the formula I in which the group —C(R⁵)═N—OR⁴ is attached in the meta- or in the para-position to the oxygen of the group O—B and among these in particular to the compounds of the formulae I-A and I-B 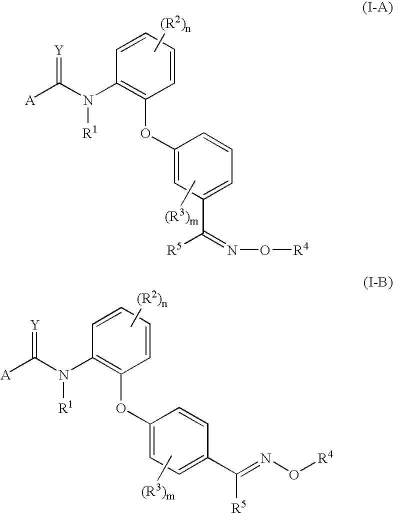
where the variables n, m, A, Y, R¹, R², R³, R⁴ and R⁵ have the meanings given above and in particular the meanings given here and below as being preferred or particularly preferred.

With a view to their fungicidal activity, preference is given to (hetero)cyclylcarbox-amides of the formula I (or I′, I-A or I-B) in which the variables Y, R¹, R², R³, R⁴, R⁵, n and m independently of one another and preferably in combination have the following meanings:
Y is O;
R¹ is hydrogen, OH, C₁-C₄-alkyl, in particular H, OH or methyl and especially H;
R² is C₁-C₄-alkyl, C₁-C₄-alkoxy, C₁-C₄-haloalkyl, C₁-C₄-haloalkoxy, nitro, cyano or halogen; particularly preferably C₁-C₄-alkyl, C₁-C₄-alkoxy, nitro, cyano or halogen and especially methyl, methoxy, fluorine, chlorine, bromine, nitro or cyano;
R³ is C₁-C₄-alkyl, C₁-C₄-alkoxy, C₁-C₄-haloalkyl, C₁-C₄-haloalkoxy, nitro, cyano or halogen; particularly preferably C₁-C₄-alkyl, C₁-C₄-alkoxy, nitro, cyano or halogen and especially methyl, methoxy, fluorine, chlorine, bromine, nitro or cyano;
n is 0 or 1, particularly preferably 0;
m is 0 or 1, particularly preferably 0;
R⁴ is C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₄-alkynyl, C₂-C₄-haloalkynyl, phenyl-C₁-C₂-alkyl or phenyl, where phenyl in the two lastmentioned radicals may be unsubstituted or may carry one or two halogen groups, especially fluorine or chlorine;
R⁵ is hydrogen, C₁-C₆-alkyl, C₁-C₆-haloalkyl, C₃-C₆-cycloalkyl, C₃-C₆-halocycloalkyl, phenyl, phenyl-C₁-C₄-alkyl, phenyl-C₁-C₄-haloalkyl, where phenyl in the three lastmentioned radicals may be unsubstituted or may carry one, two or three radicals R^{b}; preferably hydrogen, C₁-C₄-alkyl, C₁-C₄-haloalkyl, phenyl which may be unsubstituted or may carry one, two or three radicals R^{b}.

Preferred radicals R⁶ are those in which R⁷ and R⁸ independently of one another have the following meanings:
R⁷ is hydrogen, C₁-C₄-alkyl, benzyl or phenyl, where phenyl in the two lastmentioned radicals is unsubstituted or has 1 or 2 radicals R^{b};
R⁸ is C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₄-alkynyl, C₂-C₄-haloalkynyl, phenyl-C₁-C₂-alkyl or phenyl, where phenyl in the two lastmentioned radicals may be unsubstituted or may carry one or two halogen groups, especially fluorine or chlorine.

Otherwise, R^{b} is in particular halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₂-C₄-haloalkenyl or C₁-C₄-haloalkoxy.

Particular preference is furthermore given to the (heterocyclyl)carboxamides of the formula I (or I′, I-A or I-B) in which R¹, R², R³, R⁴, R⁵, n and m have the meanings given above and in particular the preferred meanings, Y is oxygen and A is selected from the group consisting of:

A-1, where X and X, are each nitrogen, R^{a1} has the meanings given above, in particular the preferred meanings and is especially methyl, trifluoromethyl, chlorine, bromine or fluorine; R^{a2} has the meanings given above and is especially hydrogen;

A-2, where X is N, W is S, R^{a1} has the meanings given above, in particular the preferred meanings and is especially methyl, fluorine, chlorine, bromine or trifluoromethyl; R^{a3} has the meanings given above, in particular the preferred meanings and is especially hydrogen;

A-2, where X is CH, W is N—R^{a4}, where R^{a4} is C₁-C₄-alkyl, especially methyl, R^{a1} has the meanings given above, in particular the preferred meanings and is especially methyl, fluorine, chlorine, bromine or trifluoromethyl; R^{a3} has the meanings given above, in particular the preferred meanings and is especially hydrogen;

A-3, where U is O, X is N, R^{a1} has the meanings given above, in particular the preferred meanings and is especially methyl, fluorine, chlorine, bromine or trifluoromethyl; R^{a3} has the meanings given above, in particular the preferred meanings and is especially hydrogen or methyl;

A-3, where U is S, X is CH, R^{a1} has the meanings given above, in particular the preferred meanings and is especially methyl, fluorine, chlorine, bromine or trifluoromethyl; R^{a3} has the meanings given above, in particular the preferred meanings and is especially hydrogen or methyl;

A-4, where U is O, X is CH or N, R^{a1} has the meanings given above, in particular the preferred meanings and is especially methyl, fluorine, chlorine, bromine or trifluoromethyl; R^{a3} has the meanings given above, in particular the preferred meanings and is especially hydrogen or methyl;

A-4, where U is S, X is CH or N, R^{a1} has the meanings given above, in particular the preferred meanings and is especially methyl, fluorine, chlorine, bromine or trifluoromethyl; R^{a3} has the meanings given above, in particular the preferred meanings and is especially hydrogen or methyl;

A-5, where U is oxygen, Z is CH₂, S, S(═O) or S(═O)₂ and R^{a1} has the meanings given above, in particular the preferred meanings and is especially methyl, fluorine, chlorine, bromine or trifluoromethyl;

A-6, where X, is nitrogen, R^{a2} has the meanings given above and is especially hydrogen; R^{a1} has the meanings given above, in particular the preferred meanings and is especially methyl, fluorine, chlorine, bromine or trifluoromethyl.

With a view to their use as fungicides, preference is given to compounds of the formula I-A where Y═O, R¹═H, n=0 and m=0 and in which the variables A, R⁴ and R⁵ have the meanings given above and in particular the meanings given as being preferred or particularly preferred (compounds I-A′). Examples of these are the compounds of the formula I-A′ compiled in tables 1 to 42 below (compounds I-A where R¹═H, n=0 and m=0), where R⁴ and R⁵ in each case have the meanings given in one row of table A and the variable A has the meaning given in the respective table. In the case of compounds which contain double bonds, both the isomerically pure E isomers, Z isomers and isomer mixtures thereof are included.

With a view to their use as fungicides, preference is given to compounds of the formula I-B where Y═O, R¹═H, n=0 and m=0 and in which the variables A, R⁴ and R⁵ have the meanings given above and in particular the meanings given as being preferred or particularly preferred (compounds I-B′). Examples of these are the compounds of the formula I—B′ compiled in tables 1 to 42 below (compounds I—B where R¹═H, n=0 and m=0), where R⁴ and R⁵ in each case have the meanings given in one row of table A and the variable A has the meaning given in the respective table. In the case of compounds which contain double bonds, both the isomerically pure E isomers, Z isomers and isomer mixtures thereof are included.

TABLE A

| | |
|---|---|
| | (I-A′) |
| 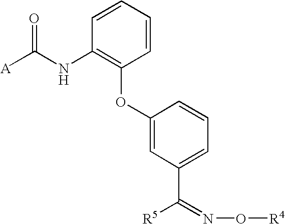 | |
| 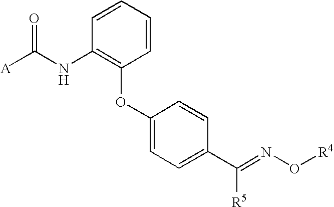 | (I-B′) |

| No. | R⁵ | R⁴ |
|---|---|---|
| 1 | H | CH₃ |
| 2 | H | C₂H₅ |
| 3 | H | CH₂CH₂CH₃ |
| 4 | H | CH(CH₃)₂ |
| 5 | H | CH₂CH₂CH₂CH₃ |
| 6 | H | i-C₄H₉ |
| 7 | H | s-C₄H₉ |
| 8 | H | C(CH₃)₃ |
| 9 | H | CH₂CH₂CH₂CH₂CH₃ |
| 10 | H | CH₂CH₂CH₂CH₂CH₂CH₃ |
| 11 | H | cyclopentyl |
| 12 | H | cyclohexyl |
| 13 | H | allyl |
| 14 | H | but-2-en-1-yl |
| 15 | H | 4-chlorobut-2-en-1-yl |
| 16 | H | propargyl |
| 17 | H | C₆H₅ |
| 18 | H | C₆H₅CH₂ |
| 19 | H | 2-phenyleth-1-yl |
| 20 | H | 4-Cl—C₆H₄ |
| 21 | H | 4-F—C₆H₄ |
| 22 | CH₃ | CH₃ |
| 23 | CH₃ | C₂H₅ |
| 24 | CH₃ | CH₂CH₂CH₃ |
| 25 | CH₃ | CH(CH₃)₂ |
| 26 | CH₃ | CH₂CH₂CH₂CH₃ |
| 27 | CH₃ | i-C₄H₉ |
| 28 | CH₃ | s-C₄H₉ |
| 29 | CH₃ | C(CH₃)₃ |
| 30 | CH₃ | CH₂CH₂CH₂CH₂CH₃ |
| 31 | CH₃ | CH₂CH₂CH₂CH₂CH₂CH₃ |
| 32 | CH₃ | cyclopentyl |
| 33 | CH₃ | cyclohexyl |
| 34 | CH₃ | allyl |
| 35 | CH₃ | but-2-en-1-yl |
| 36 | CH₃ | 4-chlorobut-2-en-1-yl |
| 37 | CH₃ | propargyl |
| 38 | CH₃ | C₆H₅ |
| 39 | CH₃ | C₆H₅CH₂ |
| 40 | CH₃ | 2-phenyleth-1-yl |
| 41 | CH₃ | 4-Cl—C₆H₄ |
| 42 | CH₃ | 4-F—C₆H₄ |
| 43 | C₂H₅ | CH₃ |
| 44 | C₂H₅ | C₂H₅ |
| 45 | C₂H₅ | CH₂CH₂CH₃ |
| 46 | C₂H₅ | CH(CH₃)₂ |
| 47 | C₂H₅ | CH₂CH₂CH₂CH₃ |
| 48 | C₂H₅ | i-C₄H₉ |
| 49 | C₂H₅ | s-C₄H₉ |
| 50 | C₂H₅ | C(CH₃)₃ |
| 51 | C₂H₅ | CH₂CH₂CH₂CH₂CH₃ |
| 52 | C₂H₅ | CH₂CH₂CH₂CH₂CH₂CH₃ |
| 53 | C₂H₅ | cyclopentyl |
| 54 | C₂H₅ | cyclohexyl |
| 55 | C₂H₅ | allyl |
| 56 | C₂H₅ | but-2-en-1-yl |
| 57 | C₂H₅ | 4-chlorobut-2-en-1-yl |
| 58 | C₂H₅ | propargyl |
| 59 | C₂H₅ | C₆H₅ |
| 60 | C₂H₅ | C₆H₅CH₂ |
| 61 | C₂H₅ | 2-phenyleth-1-yl |
| 62 | C₂H₅ | 4-Cl—C₆H₄ |
| 63 | C₂H₅ | 4-F—C₆H₄ |
| 64 | CH₂CH₂CH₃ | CH₃ |
| 65 | CH₂CH₂CH₃ | C₂H₅ |
| 66 | CH₂CH₂CH₃ | CH₂CH₂CH₃ |
| 67 | CH₂CH₂CH₃ | CH(CH₃)₂ |
| 68 | CH₂CH₂CH₃ | CH₂CH₂CH₂CH₃ |
| 69 | CH₂CH₂CH₃ | i-C₄H₉ |
| 70 | CH₂CH₂CH₃ | s-C₄H₉ |
| 71 | CH₂CH₂CH₃ | C(CH₃)₃ |
| 72 | CH₂CH₂CH₃ | CH₂CH₂CH₂CH₂CH₃ |
| 73 | CH₂CH₂CH₃ | CH₂CH₂CH₂CH₂CH₂CH₃ |
| 74 | CH₂CH₂CH₃ | cyclopentyl |
| 75 | CH₂CH₂CH₃ | cyclohexyl |
| 76 | CH₂CH₂CH₃ | allyl |
| 77 | CH₂CH₂CH₃ | but-2-en-1-yl |
| 78 | CH₂CH₂CH₃ | 4-chlorobut-2-en-1-yl |
| 79 | CH₂CH₂CH₃ | propargyl |
| 80 | CH₂CH₂CH₃ | C₆H₅ |
| 81 | CH₂CH₂CH₃ | C₆H₅CH₂ |
| 82 | CH₂CH₂CH₃ | 2-phenyleth-1-yl |
| 83 | CH₂CH₂CH₃ | 4-Cl—C₆H₄ |
| 84 | CH₂CH₂CH₃ | 4-F—C₆H₄ |
| 85 | CH(CH₃)₂ | CH₃ |
| 86 | CH(CH₃)₂ | C₂H₅ |
| 87 | CH(CH₃)₂ | CH₂CH₂CH₃ |
| 88 | CH(CH₃)₂ | CH(CH₃)₂ |
| 89 | CH(CH₃)₂ | CH₂CH₂CH₂CH₃ |
| 90 | CH(CH₃)₂ | i-C₄H₉ |
| 91 | CH(CH₃)₂ | s-C₄H₉ |
| 92 | CH(CH₃)₂ | C(CH₃)₃ |
| 93 | CH(CH₃)₂ | CH₂CH₂CH₂CH₂CH₃ |
| 94 | CH(CH₃)₂ | CH₂CH₂CH₂CH₂CH₂CH₃ |
| 95 | CH(CH₃)₂ | cyclopentyl |
| 96 | CH(CH₃)₂ | cyclohexyl |
| 97 | CH(CH₃)₂ | allyl |
| 98 | CH(CH₃)₂ | but-2-en-1-yl |
| 99 | CH(CH₃)₂ | 4-chlorobut-2-en-1-yl |
| 100 | CH(CH₃)₂ | propargyl |
| 101 | CH(CH₃)₂ | C₆H₅ |
| 102 | CH(CH₃)₂ | C₆H₅CH₂ |
| 103 | CH(CH₃)₂ | 2-phenyleth-1-yl |
| 104 | CH(CH₃)₂ | 4-Cl—C₆H₄ |
| 105 | CH(CH₃)₂ | 4-F—C₆H₄ |
| 106 | C₆H₅ | CH₃ |
| 107 | C₆H₅ | C₂H₅ |
| 108 | C₆H₅ | CH₂CH₂CH₃ |
| 109 | C₆H₅ | CH(CH₃)₂ |
| 110 | C₆H₅ | CH₂CH₂CH₂CH₃ |
| 111 | C₆H₅ | i-C₄H₉ |
| 112 | C₆H₅ | s-C₄H₉ |
| 113 | C₆H₅ | C(CH₃)₃ |
| 114 | C₆H₅ | CH₂CH₂CH₂CH₂CH₃ |
| 115 | C₆H₅ | CH₂CH₂CH₂CH₂CH₂CH₃ |
| 116 | C₆H₅ | cyclopentyl |
| 117 | C₆H₅ | cyclohexyl |
| 118 | C₆H₅ | allyl |
| 119 | C₆H₅ | but-2-en-1-yl |
| 120 | C₆H₅ | 4-chlorobut-2-en-1-yl |
| 121 | C₆H₅ | propargyl |
| 122 | C₆H₅ | C₆H₅ |
| 123 | C₆H₅ | C₆H₅CH₂ |
| 124 | C₆H₅ | 2-phenyleth-1-yl |
| 125 | C₆H₅ | 4-Cl—C₆H₄ |
| 126 | C₆H₅ | 4-F—C₆H₄ |

s-C₄H₉: —CH(CH₃)(C₂H₅);
i-C₄H₉: CH₂CH(CH₃)₂;
allyl: —CH₂CH═CH₂;
propargyl: —CH₂C≡CH;

Table 1:

Compounds of the formulae I-A′ and I-B′, where A is 2-chlorophenyl and R⁴ and R⁵ for each individual compound correspond in each case to one row of table A.

Table 2:

Compounds of the formulae I-A′ and I-B′ in which A is 2-trifluoromethylphenyl and R⁴ and R⁵ for each individual compound correspond in each case to one row of table A.

Table 3:

Compounds of the formulae I-A′ and I-B′ in which A is 2-difluoromethylphenyl and R⁴ and R⁵ for each individual compound correspond in each case to one row of table A.

Table 4:

Compounds of the formulae I-A′ and I-B′ in which A is 2-methylphenyl and R⁴ and R⁵ for each individual compound correspond in each case to one row of table A.

Table 5:

Compounds of the formulae I-A′ and I-B′ in which A is 2-chloropyridin-3-yl and R⁴ and R⁵ for each individual compound correspond in each case to one row of table A.

Table 6:

Compounds of the formulae I-A′ and I-B′ in which A is 2-trifluoromethylpyridin-3-yl and R⁴ and R⁵ for each individual compound correspond in each case to one row of table A.

Table 7:

Compounds of the formulae I-A′ and I-B′ in which A is 2-difluoromethylpyridin-3-yl and R⁴ and R⁵ for each individual compound correspond in each case to one row of table A.

Table 8:

Compounds of the formulae I-A′ and I-B′ in which A is 2-methylpyridin-3-yl and R⁴ and R⁵ for each individual compound correspond in each case to one row of table A.

Table 9:

Compounds of the formulae I-A′ and I-B′ in which A is 4-methylpyridimidin-5-yl and R⁴ and R⁵ for each individual compound correspond in each case to one row of table A.

Table 10:

Compounds of the formulae I-A′ and I-B′ in which A is 4-trifluoromethylpyrimidin-5-yl and R⁴ and R⁵ for each individual compound correspond in each case to one row of table A.

Table 11:

Compounds of the formulae I-A′ and I-B′ in which A is 4-difluoromethylpyrimidin-5-yl and R⁴ and R⁵ for each individual compound correspond in each case to one row of table A.

Table 12:

Compounds of the formulae I-A′ and I-B′ in which A is 1-methyl-3-trifluoromethylpyrazol-4-yl and R⁴ and R⁵ for each individual compound correspond in each case to one row of table A.

Table 13:

Compounds of the formulae I-A′ and I-B′ in which A is 1-methyl-3-difluoromethylpyrazol-4-yl and R⁴ and R⁵ for each individual compound correspond in each case to one row of table A.

Table 14:

Compounds of the formulae I-A′ and I-B′ in which A is 1,3-dimethylpyrazol-4-yl and R⁴ and R⁵ for each individual compound correspond in each case to one row of table A.

Table 15:

Compounds of the formulae I-A′ and I-B′ in which A is 1-methyl-3-trifluoromethyl-5-fluoropyrazol-4-yl and R⁴ and R⁵ for each individual compound correspond in each case to one row of table A.

Table 16:

Compounds of the formulae I-A′ and I-B′ in which A is 1-methyl-3-difluoromethyl-5-fluoropyrazol-4-yl and R⁴ and R⁵ for each individual compound correspond in each case to one row of table A.

Table 17:

Compounds of the formulae I-A′ and I-B′ in which A is 1-methyl-3-trifluoromethyl-5-chloropyrazol-4-yl and R⁴ and R⁵ for each individual compound correspond in each case to one row of table A.

Table 18:

Compounds of the formulae I-A′ and I-B′ in which A is 1-methyl-3-trifluoromethylpyrol-4-yl and R⁴ and R⁵ for each individual compound correspond in each case to one row of table A.

Table 19:

Compounds of the formulae I-A′ and I-B′ in which A is 1-methyl-3-difluoromethylpyrol-4-yl and R⁴ and R⁵ for each individual compound correspond in each case to one row of table A.

Table 20:

Compounds of the formulae I-A′ and I-B′ in which A is 2-methyl-4-trifluoromethylthiazol-5-yl and R⁴ and R⁵ for each individual compound correspond in each case to one row of table A.

Table 21:

Compounds of the formulae I-A′ and I-B′ in which A is 2-methyl-4-difluoromethylthiazol-5-yl and R⁴ and R⁵ for each individual compound correspond in each case to one row of table A.

Table 22:

Compounds of the formulae I-A′ and I-B′ in which A is 2,4-dimethylthiazol-5-yl and R⁴ and R⁵ for each individual compound correspond in each case to one row of table A.

Table 23:

Compounds of the formulae I-A′ and I-B′ in which A is 2-methyl-5-trifluoromethylthiazol-4-yl and R⁴ and R⁵ for each individual compound correspond in each case to one row of table A.

Table 24:

Compounds of the formulae I-A′ and I-B′ in which A is 2-methyl-5-difluoromethylthiazol-4-yl and R⁴ and R⁵ for each individual compound correspond in each case to one row of table A.

Table 25:

Compounds of the formulae I-A′ and I-B′ in which A is 2,5-dimethylthiazol-4-yl and R⁴ and R⁵ for each individual compound correspond in each case to one row of table A.

Table 26:

Compounds of the formulae I-A′ and I-B′ in which A is 2-methyl-4-trifluoromethyloxazol-5-yl and R⁴ and R⁵ for each individual compound correspond in each case to one row of table A.

Table 27:

Compounds of the formulae I-A′ and I-B′ in which A is 2-methyl-4-difluoromethyloxazol-5-yl and R⁴ and R⁵ for each individual compound correspond in each case to one row of table A.

Table 28:

Compounds of the formulae I-A′ and I-B′ in which A is 2,4-dimethyloxazol-5-yl and R⁴ and R⁵ for each individual compound correspond in each case to one row of table A.

Table 29:

Compounds of the formulae I-A′ and I-B′ in which A is 2-trifluoromethylthiophen-3-yl and R⁴ and R⁵ for each individual compound correspond in each case to one row of table A.

Table 30:

Compounds of the formulae I-A′ and I-B′ in which A is 5-methyl-2-trifluoromethyl-thiophen-3-yl and R⁴ and R⁵ for each individual compound correspond in each case to one row of table A.

Table 31:

Compounds of the formulae I-A′ and I-B′ in which A is 2-methylthiophen-3-yl and R⁴ and R⁵ for each individual compound correspond in each case to one row of table A.

Table 32:

Compounds of the formulae I-A′ and I-B′ in which A is 2,5-dimethylthiophen-3-yl and R⁴ and R⁵ for each individual compound correspond in each case to one row of table A.

Table 33:

Compounds of the formulae I-A′ and I-B′ in which A is 3-trifluoromethylthiophen-2-yl and R⁴ and R⁵ for each individual compound correspond in each case to one row of table A.

Table 34:

Compounds of the formulae I-A′ and I-B′ in which A is 3-methylthiophen-2-yl and R⁴ and R⁵ for each individual compound correspond in each case to one row of table A.

Table 35:

Compounds of the formulae I-A′ and I-B′ in which A is 3,5-dimethylthiophen-2-yl and R⁴ and R⁵ for each individual compound correspond in each case to one row of table A.

Table 36:

Compounds of the formulae I-A′ and I-B′ in which A is 5-methyl-3-trifluoromethylthiophen-2-yl and R⁴ and R⁵ for each individual compound correspond in each case to one row of table A.

Table 37:

Compounds of the formulae I-A′ and I-B′ in which A is 2-trifluoromethyfuran-3-yl and R⁴ and R⁵ for each individual compound correspond in each case to one row of table A.

Table 38:

Compounds of the formulae I-A′ and I-B′ in which A is 5-methyl-2-trifluoromethylfuran-3-yl and R⁴ and R⁵ for each individual compound correspond in each case to one row of table A.

Table 39:

Compounds of the formulae I-A′ and I-B′ in which A is 2-methylfuran-3-yl and R⁴ and R⁵ for each individual compound correspond in each case to one row of table A.

Table 40:

Compounds of the formulae I-A′ and I-B′ in which A is 2,5-dimethylfuran-3-yl and R⁴ and R⁵ for each individual compound correspond in each case to one row of table A.

Table 41:

Compounds of the formulae I-A′ and I-B′ in which A is 2-methyl-5,6-dihydro-[1,4]oxathiin-3-yl and R⁴ and R⁵ for each individual compound correspond in each case to one row of table A.

Table 42:

Compounds of the formulae I-A′ and I-B′ in which A is 2-methyl-5,6-dihydro-4H-thiopyran-3-yl and R⁴ and R⁵ for each individual compound correspond in each case to one row of table A.

The compounds of the formula I according to the invention can be prepared by prior art methods known per se, for example according to scheme 1 by reacting activated (heterocyclyl)carboxylic acid derivatives II with an aniline III [Houben-Weyl: “Methoden der organ. Chemie” [Methods of Organic Chemistry], Georg-Thieme-Verlag, Stuttgart, N.Y. 1985, Volume E5, pp. 941-[1045]. Activated carboxylic acid derivatives II are, for example, halides, activated esters, anhydrides, azides, for example chlorides, fluorides, bromides, para-nitrophenyl esters, pentafluorophenyl esters, N-hydroxysuccinimides, hydroxybenzotriazol-1-yl esters. In scheme 1, the radicals A, Y, R¹, R², R³, R⁴, R⁵, n and m have the meanings mentioned above and in particular the meanings mentioned as being preferred.

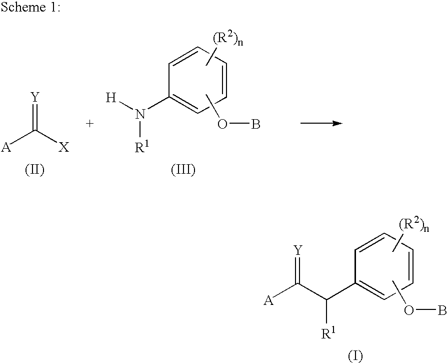

The active compounds I can also be prepared, for example, by reacting the acids IV with an aniline III in the presence of a coupling agent according to scheme 2. In scheme 2, the radicals A, Y, R¹, R², R^{3m}, R^{4m}, R⁵, R⁶, n and m have the meanings given above and in particular the meanings given as being preferred.

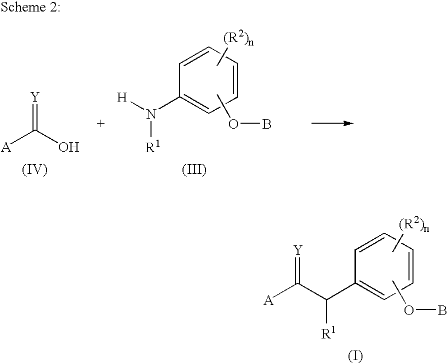

Suitable coupling agents are, for example:
coupling agents based on carbodiimides, for example N,N′-dicyclohexylcarbodiimide [J. C. Sheehan, G. P. Hess, J. Am. Chem. Soc. 1955, 77, 1067], N-(3-dimethylaminopropyl)-N′-ethylcarbodiimide;
coupling agents which form mixed anhydrides with carbonic esters, for example 2-ethoxy-1-ethoxycarbonyl-1,2-dihydroquinoline [B. Belleau, G. Malek, J. Amer. Chem. Soc. 1968, 90, 1651], 2-isobutyloxy-1-isobutyloxycarbonyl-1,2-dihydroquinoline [Y. Kiso, H. Yajima, J. Chem. Soc., Chem. Commun. 1972, 942];
coupling agents based on phosphonium salts, for example (benzotriazol-1-yloxy)tris(dimethylamino)phosphonium hexafluorophosphate [B. Castro, J. R. Domoy, G. Evin, C. Selve, Tetrahedron Lett. 1975, 14, 1219], (benzotriazol-1-yl-oxy)tripyrrolidinophosphonium hexafluorophosphate [J. Coste et al., Tetrahedron Lett. 1990, 31, 205];
coupling agents based on uronium salts or having a guanidinium N-oxide structure, for example N,N, N′,N′-tetramethyl-O-(1H-benzotriazol-1-yl)uronium hexafluorophos-phate [R. Knorr, A. Trzeciak, W. Bannwarth, D. Gillessen, Tetrahedron Lett. 1989, 30, 1927], N,N, N′,N′-tetramethyl-O-(benzotriazol-1-yl)uronium tetrafluoroborate, (benzotriazol-1-yloxy)dipiperidinocarbenium hexafluorophosphate [S. Chen, J. Xu, Tetrahedron Lett. 1992, 33, 647];
coupling agents which form acid chlorides, for example bis-(2-oxo-oxazolidinyl)phosphinic chloride [J. Diago-Mesequer, Synthesis 1980, 547].

Compounds I where R¹=optionally halogen-substituted alkyl or optionally substituted cycloalkyl can also be prepared by alkylating the amides I (in which R¹ is hydrogen and which can be obtained according to scheme 1 or 2) using suitable alkylating agents in the presence of bases, see scheme 3.

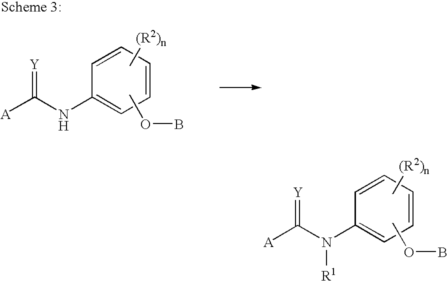

The (heterocyclyl)carboxylic acids IV can be prepared by methods known from the literature, and from these, the (heterocyclyl)carboxylic acid derivatives II can be prepared by methods known from the literature [for example EP 0589313, EP 915868, U.S. Pat. No. 4,877,441].

The anilines III can be prepared, for example, by the methods shown in scheme 4. In scheme 4, the radicals R¹, R², R³, R⁴, R⁵, n and m have the meanings given above and in particular the meanings given as being preferred. The compounds V and X are known from the literature or can be prepared by methods known from the literature. 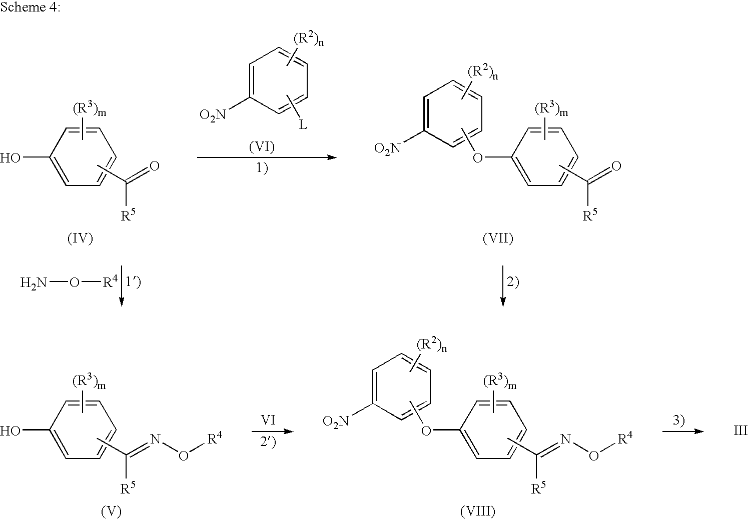
Scheme 4:

In step 1 in scheme 4, the nitroaromatic compound VI in which L is halogen, for example fluorine, chlorine or bromine, is reacted with an acylphenol IX in the sense of a nucleophilic aromatic substitution, which yields the nitrobiphenyl ether VII. The reaction is carried out analogously to known processes, for example according to Organikum, 21st edition, Wiley-VCH 2001, p. 394ff. S. Raeppel, F. Raeppel, J. Suffert; Synlett [SYNLES] 1998, (7), 794-796. R. Beugelmans, A. Bigot, J. Zhu; Tetrahedron Lett [TELEAY] 1994, 35 (31), 5649-5652. The reaction is usually carried out in the presence of a base. Suitable bases are alkali metal carbonates, alkaline earth metal carbonates, such as sodium carbonate, potassium carbonate, calcium carbonate, magnesium carbonate, alkali metal hydroxides or alkaline earth metal hydroxides, such as sodium hydroxide or potassium hydroxide. In general, the reaction is carried out in an inert organic solvent. Suitable solvents are ethers, such as diethyl ether, methyl tert-butyl ether, dioxane, tetrahydrofuran, ethylene glycol dimethyl ether, diethylene glycol.

In step 2, the nitrophenyl ether VII is reacted with a hydroxylamine H₂N—O—R⁴ or with an acid addition salt thereof, for example the hydrochloride HCl.H₂N—O—R⁴, which yields the oximated nitrobiphenyl ether VIII. The reaction is generally carried out in a solvent. Suitable solvents are, for example, C₁-C₄-alcohols or C₁-C₄-alcohol/water mixtures. The reaction can be carried out in the presence of a base. Suitable bases are aromatic amines, such as pyridine, or alkali metal hydroxides or alkaline earth metal hydroxides, such as sodium hydroxide, potassium hydroxide or calcium hydroxide. The oximation of the keto group in VII can be carried out, for example, analogously to Organikum, 21st edition, Wiley-VCH 2001, p. 467 or D. Dhanak, C. Reese, S. Romana, G. Zappia, J. Chem. Soc. Chem. Comm. 1986 (12), 903-904, DE 3004871 or AU 580091.

In a similar manner, the oximated nitrobiphenyl ether of the formula VIII can be prepared by oximating, in a first step 1′), the acylphenol compound IX analogously to step 2) by reaction with H₂N—OR⁴ and then, in step 2′), reacting the phenol V oximated in this manner with the nitroaromatic compound VI. The reaction conditions in steps 1′) and 2′) correspond essentially to the conditions given for steps 1) and 2), respectively.

In step 3, the nitrobiphenyl ether VIII obtained in step 2) or 2′) is then reduced to the aminobiphenyl ether III. The reduction is carried out by processes customary for reducing organic nitro compounds as described, for example, in Organikum, 21st edition, Wiley-VCH 2001, p. 627ff. The reduction of the nitro group of the nitrobiphenyl ether VIII is preferably carried out as a catalytic reduction over a transition metal catalyst, suitable hydrogen sources including, in addition to hydrogen, hydrazine. Suitable transition metal catalysts are, in particular heterogeneous catalysts with transition metals of group VIII, in particular with palladium, platinum or nickel as active metal, for example palladium-on-carbon or Raney nickel. The reduction is generally carried out in an inert solvent, for example a C₁-C₄-alcohol, such as methanol or ethanol. The reduction of the nitrobiphenyl ether VIII to the aminobiphenyl ether III can also be effected, for example, by reacting the nitrophenyl ether VIII with a metal compound, such as tin(II) chloride, under acidic reaction conditions such as concentrated hydrochloric acid.

The compounds I are suitable for use as fungicides. They are distinguished by an outstanding effectiveness against a broad spectrum of phytopathogenic fungi, especially from the classes of the Ascomycetes, Deuteromycetes, Phycomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, corn, grass, bananas, cotton, soybean, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Botrytis cinerea (gray mold) on strawberries, vegetables, ornamental plants and grapevines,
Cercospora arachidicola on groundnuts, Erysiphe cichoracearum and Sphaerotheca fuliginea on cucurbits,
Erysiphe graminis (powdery mildew) on cereals,
Fusarium and Verticillium species on various plants,
Helminthosporium species on cereals,
Mycosphaerella species on bananas and groundnuts,
Phytophthora infestans on potatoes and tomatoes,
Phakopsora spp. on soybean,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria nodorum on wheat,
Sphaerotheca fuliginea (mildew of cucumber) on cucumbers,
Uncinula necator on grapevines,
Ustilago species on cereals and sugar cane,
Venturia species (scab) on apples and pears,
Septoria tritici,
Pyrenophora species,
Leptosphaeria nodorum,
Rhynchosporium species and
Typhula species.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally necessary.

When used in the protection of materials or stored products, the amount of active compound applied depends on the kind of application area and on the effect desired. Amounts customarily applied in the protection of materials are, for example, 0.001 g to 2 kg, preferably 0.005 g to 1 kg, of active compound per cubic meter of treated material.

The compounds I can be converted to the usual formulations, e.g. solutions, emulsions, suspensions, dusts, powders, pastes and granules. The application form depends on the respective use intended; it should in any case guarantee a fine and uniform distribution of the compound according to the invention.

The formulations are prepared in a known way, e.g. by extending the active compound with solvents and/or carriers, if desired using emulsifiers and dispersants, it being possible, when water is the diluent, also to use other organic solvents as auxiliary solvents. Suitable auxiliaries for this purpose are essentially: solvents, such as aromatics (e.g. xylene), chlorinated aromatics (e.g. chlorobenzenes), paraffins (e.g. petroleum fractions), alcohols (e.g. methanol, butanol), ketones (e.g. cyclohexanone), amines (e.g. ethanolamine, dimethylformamide) and water; carriers, such as ground natural minerals (e.g. kaolins, clays, talc, chalk) and ground synthetic ores (e.g. highly dispersed silicic acid, silicates); emulsifiers, such as nonionic and anionic emulsifiers (e.g. polyoxyethylene fatty alcohol ethers, alkylsulfonates and arylsulfonates) and dispersants, such as lignosulfite waste liquors and methylcellulose.

Suitable surfactants are alkali metal, alkaline earth metal and ammonium salts of lignosulfonic acid, naphthalenesulfonic acid, phenolsulfonic acid and dibutylnaphthalenesulfonic acid, alkylarylsulfonates, alkyl sulfates, alkylsulfonates, fatty alcohol sulfates and fatty acids, and alkali metal and alkaline earth metal salts thereof, salts of sulfated fatty alcohol glycol ethers, condensation products of sulfonated naphthalene and naphthalene derivatives with formaldehyde, condensation products of naphthalene or of naphthalenesulfonic acid with phenol and formaldehyde, polyoxyethylene octylphenol ethers, ethoxylated isooctylphenol, octylphenol and nonylphenol, alkylphenol polyglycol ethers, tributylphenyl polyglycol ethers, alkylaryl polyether alcohols, isotridecyl alcohol, fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Petroleum fractions having medium to high boiling points, such as kerosene or diesel fuel, furthermore coal tar oils, and oils of vegetable or animal origin, aliphatic, cyclic and aromatic hydrocarbons, e.g. benzene, toluene, xylene, paraffin, tetrahydronaphthalene, alkylated naphthalenes or derivatives thereof, methanol, ethanol, propanol, butanol, chloroform, carbon tetrachloride, cyclohexanol, cyclohexanone, chlorobenzene or isophorone, or highly polar solvents, e.g. dimethylformamide, dimethyl sulfoxide, N-methylpyrrolidone or water, are suitable for the preparation of directly sprayable solutions, emulsions, pastes or oil dispersions.

Powders, combinations for broadcasting and dusts can be prepared by mixing or mutually grinding the active substances with a solid carrier.

Granules, e.g. coated granules, impregnated granules and homogeneous granules, can be prepared by binding the active compounds to solid carriers. Solid carriers are, e.g., mineral earths, such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate or ureas, and plant products, such as cereal meal, tree bark meal, wood meal and nutshell meal, cellulose powders and other solid carriers.

The formulations generally comprise between 0.01 and 95% by weight, preferably between 0.1 and 90% by weight, of the active compound. The active compounds are employed therein in a purity of 90% to 100%, preferably 95% to 100% (according to the NMR spectrum).

Examples for Formulations are:
I. 5 parts by weight of a compound according to the invention are intimately mixed with 95 parts by weight of finely divided kaolin. In this way, a dust comprising 5% by weight of the active compound is obtained.
II. 30 parts by weight of a compound according to the invention are intimately mixed with a mixture of 92 parts by weight of pulverulent silica gel and 8 parts by weight of liquid paraffin, which had been sprayed onto the surface of this silica gel. In this way, an active compound preparation with good adhesive properties (active compound content 23% by weight) is obtained.
III. 10 parts by weight of a compound according to the invention are dissolved in a mixture consisting of 90 parts by weight of xylene, 6 parts by weight of the addition product of 8 to 10 mol of ethylene oxide with 1 mol of the N-mono-ethanolamide of oleic acid, 2 parts by weight of the calcium salt of dodecyl-benzenesulfonic acid and 2 parts by weight of the addition product of 40 mol of ethylene oxide with 1 mol of castor oil (active compound content 9% by weight).
IV. 20 parts by weight of a compound according to the invention are dissolved in a mixture consisting of 60 parts by weight of cyclohexanone, 30 parts by weight of isobutanol, 5 parts by weight of the addition product of 7 mol of ethylene oxide with 1 mol of isooctylphenol and 5 parts by weight of the addition product of 40 mol of ethylene oxide with 1 mol of castor oil (active compound content 16% by weight).
V. 80 parts by weight of a compound according to the invention are intimately mixed with 3 parts by weight of the sodium salt of diisobutylnaphthalene-α-sulfonic acid, 10 parts by weight of the sodium salt of a lignosulfonic acid from a sulfite waste liquor and 7 parts by weight of pulverulent silica gel and are ground in a hammer mill (active compound content 80% by weight).
VI. 90 parts by weight of a compound according to the invention are mixed with 10 parts by weight of N-methyl-α-pyrrolidone and a solution is obtained which is suitable for use in the form of very small drops (active compound content 90% by weight).
VII. 20 parts by weight of a compound according to the invention are dissolved in a mixture consisting of 40 parts by weight of cyclohexanone, 30 parts by weight of isobutanol, 20 parts by weight of the addition product of 7 mol of ethylene oxide with 1 mol of isooctylphenol and 10 parts by weight of the addition product of 40 mol of ethylene oxide with 1 mol of castor oil. By running the solution into 100 000 parts by weight of water and finely dispersing it therein, an aqueous dispersion is obtained comprising 0.02% by weight of the active compound.
VIII. 20 parts by weight of a compound according to the invention are intimately mixed with 3 parts by weight of the sodium salt of diisobutylnaphthalene-α-sulfonic acid, 17 parts by weight of the sodium salt of a lignosulfonic acid from a sulfite waste liquor and 60 parts by weight of pulverulent silica gel and are ground in a hammer mill. A spray emulsion comprising 0.1% by weight of the active compound is obtained by fine dispersion of the mixture in 20 000 parts by weight of water.
IX. 10 parts by weight of the compound according to the invention are dissolved in 63 parts by weight of cyclohexanone, 27 parts by weight of dispersing agent (for example a mixture of 50 parts by weight of the adduct of 7 mol of ethylene oxide to 1 mol of isooctylphenol and 50 parts by weight of the adduct of 40 mol of ethylene oxide to 1 mol of castor oil). The stock solution is then diluted to the desired concentration, for example to a concentration in the range from 1 to 100 ppm, by distribution in water.

The active compounds can be used as such, in the form of their formulations or of the application forms prepared therefrom, e.g. in the form of directly sprayable solutions, powders, suspensions or dispersions, emulsions, oil dispersions, pastes, dusts, compositions for broadcasting or granules, by spraying, atomizing, dusting, broadcasting or watering. The application forms depend entirely on the intended uses; they should in any case guarantee the finest possible dispersion of the active compounds according to the invention.

Aqueous application forms can be prepared from emulsion concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, concentrates comprising active substance, wetting agent, tackifier, dispersant or emulsifier and possibly solvent or oil can also be prepared, which concentrates are suitable for dilution with water.

The concentrations of active compound in the ready-for-use preparations can be varied within relatively wide ranges. In general, they are between 0.0001 and 10%. Often even small amounts of active compound I are sufficient in the ready-to use preparation, for example 2 to 200 ppm. Ready-to-use preparations with concentrations of active compound in the range from 0.01 to 1% are also preferred.

The active compounds can also be used with great success in the ultra low volume (ULV) process, it being possible to apply formulations with more than 95% by weight of active compound or even the active compound without additives.

Oils of various types, herbicides, fungicides, other pesticides and bactericides can be added to the active compounds, if need be also not until immediately before use (tank mix). These agents can be added to the compositions according to the invention in a weight ratio of 1:10 to 10:1.

The compositions according to the invention can, in the application form as fungicides, also be present together with other active compounds, e.g. with herbicides, insecticides, growth regulators, fungicides or also with fertilizers. On mixing the compounds I or the compositions comprising them in the application form as fungicides with other fungicides, in many cases an expansion of the fungicidal spectrum of activity is obtained.

The following list of fungicides, with which the compounds according to the invention can be used in conjunction, is intended to illustrate the possible combinations but does not limit them:
sulfur, dithiocarbamates and their derivatives, such as iron(III) dimethyldithiocarbamate, zinc dimethyldithiocarbamate, zinc ethylenebisdithiocarbamate, manganese ethylenebisdithiocarbamate, manganese zinc ethylenediaminebisdithiocarbamate, tetramethylthiuram disulfide, ammonia complex of zinc (N,N′-ethylenebisdithiocarbamate), ammonia complex of zinc (N,N′-propylenebisdithiocarbamate), zinc (N,N′-propylenebisdithiocarbamate) or N,N′-polypropylenebis(thiocarbamoyl)disulfide;
nitro derivatives, such as dinitro(1-methylheptyl)phenyl crotonate, 2-sec-butyl-4,6-dinitrophenyl 3,3-dimethylacrylate, 2-sec-butyl-4,6-dinitrophenyl isopropyl carbonate or diisopropyl 5-nitroisophthalate;
heterocyclic substances, such as 2-heptadecyl-2-imidazoline acetate, 2,4-dichloro-6-(o-chloroanilino)-s-triazine, O,O-diethyl phthalimidophosphonothioate, 5-amino-1-[bis(dimethylamino)phosphinyl]-3-phenyl-1,2,4-triazole, 2,3-dicyano-1,4-dithioanthraquinone, 2-thio-1,3-dithiolo[4,5-b]quinoxaline, methyl 1-(butylcarbamoyl)-2-benzimidazolecarbamate, 2-(methoxycarbonylamino)benzimidazole, 2-(2-furyl)benzimidazole, 2-(4-thiazolyl)benzimidazole, N-(1,1,2,2-tetrachloroethylthio)tetrahydrophthalimide, N-(trichloromethylthio)tetrahydrophthalimide or N-(trichloromethylthio)phthalimide,
N-dichlorofluoromethylthio-N′,N′-dimethyl-N-phenylsulfamide, 5-ethoxy-3-trichloromethyl-1,2,3-thiadiazole, 2-thiocyanatomethylthiobenzothiazole, 1,4-dichloro-2,5-dimethoxybenzene, 4-(2-chlorophenylhydrazono)-3-methyl-5-isoxazolone, 2-thiopyridine 1-oxide, 8-hydroxyquinoline or its copper salt, 2,3-dihydro-5-carboxanilido-6-methyl-1,4-oxathiin, 2,3-dihydro-5-carboxanilido-6-methyl-1,4-oxathiin 4,4-dioxide, 2-methyl-5,6-dihydro-4H-pyran-3-carboxanilide, 2-methylfuran-3-carboxanilide, 2,5-dimethylfuran-3-carboxanilide, 2,4,5-trimethyl-furan-3-carboxanilide, N-cyclohexyl-2,5-dimethylfuran-3-carboxamide, N-cyclohexyl-N-methoxy-2,5-dimethylfuran-3-carboxamide, 2-methylbenzanilide, 2-iodobenzanilide, N-formyl-N-morpholine 2,2,2-trichloroethyl acetal, piperazine-1,4-diylbis-1-(2,2,2-trichloroethyl)formamide, 1-(3,4-dichloroanilino)-1-formyl-amino-2,2,2-trichloroethane, 2,6-dimethyl-N-tridecylmorpholine or its salts, 2,6-dimethyl-N-cyclododecylmorpholine or its salts, N-[3-(p-(tert-butyl)phenyl)-2-methylpropyl]-cis-2,6-dimethylmorpholine, N-[3-(p-(tert-butyl)phenyl)-2-methyl-propyl]piperidine, 1-[2-(2,4-dichlorophenyl)-4-ethyl-1,3-dioxolan-2-ylethyl]-1H-1,2,4-triazole, 1-[2-(2,4-dichlorophenyl)-4-(n-propyl)-1,3-dioxolan-2-ylethyl]-1H-1,2,4-triazole, N-(n-propyl)-N-(2,4,6-trichlorophenoxyethyl)-N′-imidazolylurea, 1-(4-chlorophenoxy)-3,3-dimethyl-1-(1H-1,2,4-triazol-1-yl)-2-butanone, 1-(4-chlorophenoxy)-3,3-dimethyl-1-(1H-1,2,4-triazol-1-yl)-2-butanol, (2RS,3RS)-1-[3-(2-chlorophenyl)-2-(4-fluorophenyl)oxiran-2-ylmethyl]-1H-1,2,4-triazole, α-(2-chlorophenyl)-α-(4-chlorophenyl)-5-pyrimidine methanol, 5-butyl-2-dimethylamino-4-hydroxy-6-methylpyrimidine, bis(p-chlorophenyl)-3-pyridinemethanol, 1,2-bis(3-ethoxycarbonyl-2-thioureido)benzene or 1,2-bis(3-methoxycarbonyl-2-thioureido)benzene,
strobilurins, such as methyl E-methoxyimino[α-(o-tolyloxy)-o-tolyl]acetate, methyl E-2-{2-[6-(2-cyanophenoxy)pyrimidin-4-yloxy]phenyl}-3-methoxyacrylate, methyl E-methoxyimino-[α-(2-phenoxyphenyl)]acetamide, methyl E-methoxyimino-[α-(2,5-dimethylphenoxy)-o-tolyl]acetamide,
anilinopyrimidines, such as N-(4,6-dimethylpyrimidin-2-yl)aniline, N-[4-methyl-6-(1-propynyl)pyrimidin-2-yl]aniline or N-[4-methyl-6-cyclopropylpyrimidin-2-yl]-aniline,
phenylpyrroles, such as 4-(2,2-difluoro-1,3-benzodioxol-4-yl)pyrrole-3-carbonitrile,
cinnamamides, such as 3-(4-chlorophenyl)-3-(3,4-dimethoxyphenyl)acryloyl-morpholine,
and various fungicides, such as dodecylguanidine acetate, 3-[3-(3,5-dimethyl-2-oxycyclohexyl)-2-hydroxyethyl]glutarimide, hexachlorobenzene, methyl N-(2,6-dimethylphenyl)-N-(2-furoyl)-DL-alaninate, N-(2,6-dimethylphenyl)-N-(2′-methoxyacetyl)-DL-alanine methyl ester, N-(2,6-dimethylphenyl)-N-chloroacetyl-D,L-2-aminobutyrolactone, N-(2,6-dimethylphenyl)-N-(phenylacetyl)-DL-alanine methyl ester, 5-methyl-5-vinyl-3-(3,5-dichlorophenyl)-2,4-dioxo-1,3-oxazolidine, 3-(3,5-dichlorophenyl)-5-methyl-5-methoxymethyl-1,3-oxazolidine-2,4-dione, 3-(3,5-dichlorophenyl)-1-isopropylcarbamoylhydantoin, N-(3,5-dichlorophenyl)-1,2-dimethylcyclopropane-1,2-dicarboximide, 2-cyano-N-(ethylaminocarbonyl)-2-[methoxyimino]acetamide, 1-[2-(2,4-dichlorophenyl)pentyl]-1H-1,2,4-triazole, 2,4-difluoro-α-(1H-1,2,4-triazolyl-1-methyl)benzhydryl alcohol, N-(3-chloro-2,6-dinitro-4-trifluoromethylphenyl)-5-trifluoromethyl-3-chloro-2-aminopyridine, 1-((bis(4-fluorophenyl)methylsilyl)methyl)-1H-1,2,4-triazole.

### PREPARATION EXAMPLES

### Example 1

### 2-Chloro-N-{2-[4-(1-methoximinoethyl)phenoxy)phenyl)nicotinamide

### 1.1 2-(4-Acetylphenoxy)nitrobenzene

4.1 g of 4-acetylphenol, 4.2 g of 2-fluoronitrobenzene and 8.3 g of potassium carbonate were added to 100 ml of absolute dimethylformamide, the mixture was stirred at 70° C. for 3 h and 500 ml of dilute aqueous sodium chloride solution were then added and the mixture was extracted three times with methyl tert.-butyl ether. The combined organic phases were washed twice with water and dried over sodium sulfate, and the solution was evaporated to dryness under reduced pressure. The residue was washed with pentane and dried. This gave 7.2 g of the title compound as a colorless powder.

### 1.2 2-[4-[1-Methoxyiminoethyl)phenoxy]nitrobenzene

A mixture of 7.0 g of 2-(4-acetylphenoxy)nitrobenzene, 3.4 g of hydroxylaminomethyl ether hydrochloride and 3.2 g of pyridine in 80 ml of absolute methanol was stirred at 23° C. for 17 h, and 500 ml of water were then added to the reaction mixture. The resulting mixture was extracted twice with methyl tert-butyl ether, and the organic phases were washed twice with 5% by weight strength aqueous hydrochloric acid, dried over sodium sulfate and then concentrated under reduced pressure. This gave 7.5 g of the title compound of melting point 38 to 39° C.

### 1.3 2-[4-[1-Methoxyiminoethyl)phenoxy]aniline

7.3 g of 2-[4-[1-methoxyiminoethyl)phenoxy]nitrobenzene were dissolved in 90 ml of absolute tetrahydrofuran, 0.8 g of palladium-on-carbon (10%) was added and the mixture was stirred under an atmosphere of hydrogen for 5 h. The mixture was filtered and the filtrate was concentrated under reduced pressure, which gave 6.5 g of the title compound as a brown resin.

### 1.4 2-Chloro-N-{2-[4-(1-methoximinoethyl)phenoxy)phenyl)nicotinamide

A solution of 0.51 g of 2-[4-(1-methoxyiminoethyl)phenoxy]aniline, 0.32 g of 2-chloronicotinic acid (2-chloropyridine-3-carboxylic acid), 0.3 g of triethylamine and 0.64 g of bis(2-oxo-3-oxazolidinyl)phosphoryl chloride) in 15 ml of absolute tetrahydrofuran was stirred at 23° C. for 17 h, and 20 ml of methyl tert-butyl ether were then added to the reaction mixture. The organic phase was washed twice with 5% strength aqueous sodium hydroxide solution and 5% strength hydrochloric acid, dried over sodium sulfate and concentrated under reduced pressure. Chromatographic purification gave 0.64 g of the title compound as a colorless resin.

The compounds, listed in table B, of the formula I-A or I-B where n=m=0 (Examples 2 to 12) were prepared in an analogous manner.

TABLE B

| | | | | | | m.p. [° C.] ¹⁾ | |
|---|---|---|---|---|---|---|---|
| No. | A | R¹ | R⁴ | R⁵ | formula | consistency | spectroscopic data ²⁾ |
| 1 | 2-chloropyridin- | H | CH₃ | CH₃ | I-B | resin | ¹H-NMR (CDCl₃), δ |
| | 3-yl | | | | | | [ppm]: 2.22 (s 3H), |
| | | | | | | | 3.98 (s, 3H), 6.92-7.65 |
| | | | | | | | (m, 8H), 8.17-8.62 (m, |
| | | | | | | | 3H), 8.95 (br. s, 1H) |
| 2 | 2-methyl-4- | H | CH₃ | CH₃ | I-B | oil | ¹H-NMR (CDCl₃), δ |
| | trifluoromethyl- | | | | | | [ppm]: 2.21 (s 3H), |
| | thiazol-5-yl | | | | | | 2.73 (s, 3H), 3.98 (s, |
| | | | | | | | 3H), 6.88-7.25 (m, 5H), |
| | | | | | | | 7.61-7.69 (m, 2H), |
| | | | | | | | 8.45-8.56 (m, 2H) |
| 3 | 1-methyl-3- | H | CH₃ | CH₃ | I-B | 99-100 | — |
| | trifluoromethyl- | | | | | | |
| | pyrazol-4-yl | | | | | | |
| 4 | 1-methyl-3- | H | C₂H₅ | CH₃ | I-B | 99-100 | — |
| | trifluoromethyl- | | | | | | |
| | pyrazol-4-yl | | | | | | |
| 5 | 2-methyl-4- | H | C₂H₅ | CH₃ | I-B | 62-63 | — |
| | trifluoromethyl- | | | | | | |
| | thiazol-5-yl | | | | | | |
| 6 | 2-chloropyridin- | H | C₂H₅ | CH₃ | I-B | 77-79 | — |
| | 3-yl | | | | | | |
| 7 | 1-methyl-3- | H | CH₃ | CH₃ | I-A | 96-97 | — |
| | trifluoromethyl- | | | | | | |
| | pyrazol-4-yl | | | | | | |
| 8 | 2-methyl-4- | H | CH₃ | CH₃ | I-A | — | ¹H-NMR (CDCl₃), δ |
| | trifluoromethyl- | | | | | | [ppm]: 2.19 (s, 3H), |
| | thiazol-5-yl | | | | | | 2.73 (s, 3H), 3.98 (s, |
| | | | | | | | 3H), 6.83-7.50 (m, 7H), |
| | | | | | | | 8.40-8.58 (m, 2H) |
| 9 | 2-chloropyridin- | H | CH₃ | CH₃ | I-A | — | ¹H-NMR (CDCl₃), δ |
| | 3-yl | | | | | | [ppm]: 2.18 (s, 3H), |
| | | | | | | | 3.97 (s, 3H), 6.88-7.45 |
| | | | | | | | (m, 8H), 8.18-8.61 (m, |
| | | | | | | | 3H), 8.94 (br.s., 1H) |
| 10 | 1-methyl-3- | H | C₂H₅ | CH₃ | I-A | 102-104 | ¹H-NMR (CDCl₃), δ |
| | trifluoromethyl- | | | | | | [ppm]: 1.33 (t, 3H), |
| | pyrazol-4-yl | | | | | | 2.20 (s, 3H), 3.95 (s, |
| | | | | | | | 3H), 4.25 (q, 2H), 6.87- |
| | | | | | | | 7.46 (m, 7H), 7.93 (s. |
| | | | | | | | 1H), 8.40-8.58 (m, 2H) |
| 11 | 2-methyl-4- | H | C₂H₅ | CH₃ | I-A | — | ¹H-NMR (CDCl₃), δ |
| | trifluoromethyl- | | | | | | [ppm]: 1.31 (t, 3H), |
| | thiazol-5-yl | | | | | | 2.21 (s, 3H), 2.77 (s, |
| | | | | | | | 3H), 4.22 (q, 2H), 6.87- |
| | | | | | | | 7.48 (m, 7H), 8.45-8.60 |
| | | | | | | | (m, 2H) |
| 12 | 2-chloropyridin- | H | C₂H₅ | CH₃ | I-A | — | ¹H-NMR (CDCl₃), δ |
| | 3-yl | | | | | | [ppm]: 1.32 (t, 3H), |
| | | | | | | | 2.19 (s, 3H), 4.23 (q, |
| | | | | | | | 2H), 6.87-7.45 (m, 8H), |
| | | | | | | | 8.18-8.95 (m, 4H) |

¹⁾ m.p.: melting point
²⁾ s: singlet; t: triplet; q: quartet; m: multiplet; br.s. broad singlet

### Use Examples

The active compounds were prepared as a stock solution comprising 0.25% by weight of active compound in acetone or dimethyl sulfoxide (DMSO). 1% by weight of the emulsifier Uniperol® EL (wetting agent having emulsifying and dispersing action based on ethoxylated alkylphenols) was added to this solution, and the mixture was diluted with water to the desired concentration.

### Use Example 1

### Activity Against Gray Mold on Bell Pepper Leaves Caused by Botrytis cinerea, Protective Application

Bell pepper seedlings of the cultivar “Neusiedler Ideal Elite” were, after 2 to 3 leaves were well-developed, sprayed to runoff point with an aqueous suspension having the concentration of active compound stated below. The next day, the treated plants were inoculated with a spore suspension of Botrytis cinerea in a 2% aqueous biomalt solution having a density of 0.17×10⁶ spores/ml. The test plants were then placed in a climatized chamber at temperatures between 22 and 24° C. and high atmospheric humidity. After 5 days, the extent of the fungal infection of the leaves was determined visually in %.

In this test, the plants which had been treated with 250 ppm of the active compound from example 3, example 4, example 7, example 10 or example 12 of table B showed an infection of at most 5% and the plants which had been treated with 300 ppm of the active compound from example 1, example 2 or example 6 of table B showed an infection of at most 20%, whereas the untreated plants were 90% infected.

### Use Example 2

### Curative Activity Against Brown Rust of Wheat Caused by Puccinia recondita

Leaves of potted wheat seedlings of the cultivar “Kanzler” were dusted with spores of brown rust (Puccinia recondita). The pots were then placed in a chamber with high atmospheric humidity (90 to 95%), at 20-22° C., for 24 hours. During this time, the spores germinated and the germinal tubes penetrated into the leaf tissue. The next day, the infected plants were sprayed to runoff point with an aqueous suspension having the concentration of active compound stated below. The suspension or emulsion was prepared as described above. After the spray coating had dried on, the test plants were cultivated in a greenhouse at temperatures of between 20 and 22° C. and at a relative atmospheric humidity of 65 to 70% for 7 days. The extent of the development of the rust fungus on the leaves was then determined.

In this test, the plants which had been treated with 250 ppm of the active compound from example 1, example 2, example 3, example 7, example 8, example 9, example 10, example 11 or example 12 of table 1 showed an infection of at most 10% and the plants which had been treated with 250 ppm of the active compound from example 4 of table B showed an infection of at most 20%, whereas the untreated plants were 70% infected.

### Use Example 3

### Protective Activity Against Brown Rust of Wheat Caused by Puccinia recondita

Leaves of potted wheat seedlings of the cultivar “Kanzler” were sprayed to runoff point with an aqueous suspension having the concentration of active compounds stated below. The next day, the treated plants were inoculated with a spore suspension of brown rust (Puccinia recondite). The pots were then placed in a chamber with high atmospheric humidity (90 to 95%) and at 20 to 22° C. for 24 hours. During this time, the spores germinated and the germ tubes penetrated into the leaf tissue. The next day, the plants were returned to the greenhouse and cultivated at temperatures between 20 and 22° C. and at 65 to 70% relative atmospheric humidity for 7 days. The extent of the rust fungus development on the leaves was then determined.

In this test, the plants which had been treated with 250 ppm of the active compound from example 8 or example 11 of table B showed an infection of at most 10%, whereas the untreated plants were 90% infected.

1. A (hetero)cyclyl(thio)carboxanilide of the formula I 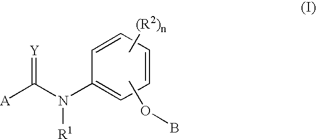
in which the variables are as defined below:
A is phenyl or an at least monounsaturated 5- or 6-membered heterocycle having 1, 2 or 3 heteroatoms selected from the group consisting of N, O, S, S(═O) and S(═O)₂ as ring members, where phenyl and the at least monounsaturated 5- or 6-membered heterocycle may be unsubstituted or may carry 1, 2 or 3 radicals R^{a}, where
R^{a} is halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl, C₁-C₄-haloalkoxy or phenyl, where phenyl may be unsubstituted or carries one, two or three radicals R^{b} selected from the group consisting of halogen, nitro, CN, C₁-C₄-alkyl, C₃-C₆-cycloalkyl, C₂-C₄-alkenyl, C₂-C₄-alkynyl, C₁-C₄-alkoxy, C₁-C₄-haloalkyl, C₃-C₆-halocycloalkyl, C₂-C₄-haloalkenyl, C₂-C₄-haloalkynyl and C₁-C₄-haloalkoxy;
B is a radical of the formula 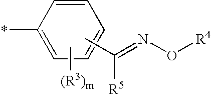
Y is oxygen or sulfur;
R¹ is H, OH, C₁-C₄-alkyl, C₃-C₆-cycloalkyl, C₁-C₄-alkoxy, C₁-C₄-haloalkyl, C₃-C₆-halocycloalkyl or C₁-C₄-haloalkoxy;
R², R³ independently of one another are halogen, nitro, CN, C₁-C₄-alkyl, C₃-C₆-cycloalkyl, C₂-C₄-alkenyl, C₂-C₄alkynyl, C₁-C₄-alkoxy, C₁-C₄-haloalkyl, C₃-C₆-halocycloalkyl, C₂-C₄-haloalkenyl, C₂-C₄-haloalkynyl or C₁-C₄-haloalkoxy;
R⁴ is hydrogen, C₁-C₈-alkyl, C₃-C₆cycloalkyl, C₂-C₈-alkenyl, C₂-C₈-alkynyl, C₁-C₈-haloalkyl, C₃-C₆-halocycloalkyl, C₂-C₈-haloalkenyl, C₂-C₈-haloalkynyl, phenyl, naphthyl, phenyl-C₁-C₄-alkyl, naphthyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl or phenyl-C₂-C₄-haloalkynyl, where phenyl and naphthyl in the 9 lastmentioned groups may be unsubstituted or may carry 1, 2 or 3 substituents selected from the group consisting of R^{b} and R⁶,
where R⁶ is —(CR⁷)═NOR⁸ in which
R⁷ is hydrogen, C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, benzyl; where phenyl and the phenyl group in benzyl may be unsubstituted or may carry one, two or three radicals R^{b}; and
R⁸ is C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, phenyl-C₁-C₄-alkyl, phenyl-C₁-C₄-haloalkyl, phenyl-C₂-C₄-alkenyl, phenyl-C₂-C₄-haloalkenyl, phenyl-C₂-C₄-alkynyl, phenyl-C₂-C₄-haloalkynyl, where phenyl in the 7 lastmentioned radicals may be unsubstituted or may carry one, two or three radicals R^{b};
R⁵ is hydrogen, C₁-C₆-alkyl, C₃-C₆-cycloalkyl, C₂-C₆-alkenyl, C₂-C₆-alkynyl, C₁-C₆-haloalkyl, C₃-C₆-halocycloalkyl, C₂-C₆-haloalkenyl, C₂-C₆-haloalkynyl, phenyl, phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl or phenyl-C₂-C₄-haloalkynyl, where phenyl in the 7 lastmentioned radicals may be unsubstituted or may carry one, two or three radicals R^{b};
n is 0, 1, 2, 3 or 4; and
m is 0, 1, 2 or 3;
or an agriculturally useful salt thereof, with the exception of compounds of the formula I in which A is 4-pyridyl.

2. A (hetero)cyclylcarboxanilide of the formula I in which A is a radical of the formula 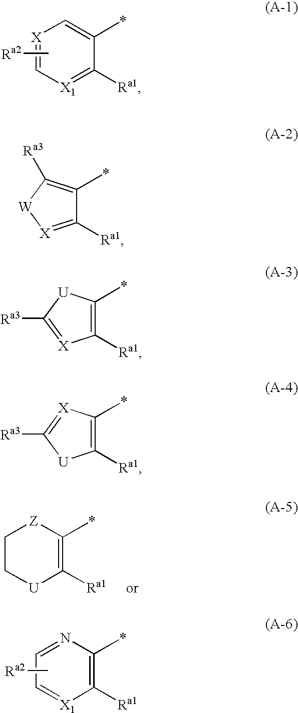
in which * denotes the point of attachment to C(═Y) and the variables are as defined below:
X, X₁ in each case independently of one another are N or CR^{c}, where R^{c} is H or has the meanings given for R^{b};
W is S or N—R^{a4} in which R^{a4} is hydrogen, C₁-C₄-alkyl, C₁-C₄-haloalkyl, C₁-C₄-alkoxy, C₁-C₄-haloalkyl or phenyl which may be unsubstituted or may carry 1, 2 or 3 radicals R^{b};
U is oxygen or sulfur;
Z is S, S(═O), S(═O)₂ or CH₂
R^{a1} is hydrogen, C₁-C₄-alkyl, C₁-C₄-alkoxy, C₁-C₄-haloalkyl, C₁-C₄-haloalkoxy or halogen;
R^{a2} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 lastmentioned groups may be substituted by halogen; and
R^{a3} is hydrogen, halogen, nitro, CN, C₁-C₄-alkyl, C₃-C₆-cycloalkyl, C₂-C₄-alkenyl, C₂-C₄-alkynyl, C₁-C₄-alkoxy, where the 5 lastmentioned groups may be substituted by halogen.

3. The (hetero)cyclylcarboxanilide of the formula I according to claim 2 in which R^{a1} is hydrogen, halogen, C₁-C₂-alkyl, C₁-C₂-alkoxy or C₁-C₂-fluoroalkyl and in which * denotes the point of attachment to C(═Y).

4. The (hetero)cyclylcarboxanilide of the formula I according to claim 2 in which A is a radical of the formula A-1a, A-2a or A-3a 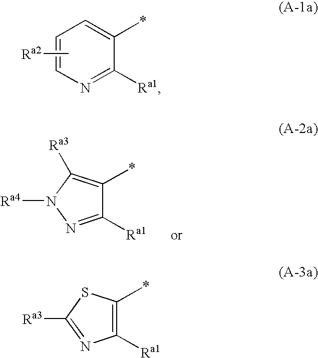
in which R^{a1}, R^{a2}, R^{a3} and R^{a4} have the meanings given in claim 2.

5. The (hetero)cyclylcarboxanilide of the formula I according to claim 4 in which A is a radical A-1a where R^{a1} halogen and R^{a2}=hydrogen or A is a radical A-2a where R^{a1}═C₁-C₂-fluoroalkyl, R^{a3}=hydrogen and R^{a4}═C₁-C₄-alkyl or A is a radical A-3a where R^{a1}═C₁-C₂-fluoroalkyl and R^{a3}═C₁-C₄-alkyl.

6. The (hetero)cyclylcarboxanilide of the formula I according to claim 1 in which R¹ is hydrogen.

7. The (hetero)cyclylcarboxanilide of the formula I according to claim 1 in which R² is C₁-C₄-alkyl, C₁-C₄-alkoxy, C₁-C₄-haloalkyl, C₁-C₄-haloalkoxy, nitro, cyano or halogen.

8. The (hetero)cyclylcarboxanilide of the formula I according to claim 1 in which n is 0 or 1.

9. The (hetero)cyclylcarboxanilide of the formula I according to claim 1 in which R⁵ is hydrogen, C₁-C₆-alkyl, C₁-C₆-haloalkyl, C₃-C₆-cycloalkyl, C₃-C₆-halocycloalkyl, phenyl, phenyl-C₁-C₄-alkyl, phenyl-C₁-C₄-haloalkyl, where phenyl in the three lastmentioned radicals may be unsubstituted or may carry one, two or three radicals R^{b}.

10. The (hetero)cyclylcarboxanilide of the formula I according to claim 1 in which R⁵ is C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₄-alkynyl, C₂-C₄-haloalkynyl, phenyl-C₁-C₂-alkyl or phenyl, where phenyl in the two lastmentioned radicals may be unsubstituted or may carry one or two halogen groups.

11. The (hetero)cyclylcarboxanilide of the formula I according to claim 1 in which Y is oxygen.

12. The (hetero)cyclylcarboxanilide of the formula I according to claim 1 in which the group O—B is attached in the ortho-position to the group N—R¹.

13. The (hetero)cyclylcarboxanilide of the formula I according to claim 1 in which the group —C(R⁵)═N—OR⁴ is attached in the meta- or para-position to the oxygen of the group O—B.

14. The (hetero)cyclylcarboxanilide of the formula I according to claim 1 in which m is 0 or 1.

15. The use of a (hetero)cyclyl(thio)carboxanilide of the formula I according to claim 1 or of an agriculturally useful salt thereof for controlling harmful fungi.

16. A crop protection composition comprising at least one (hetero)cyclyl(thio)carboxanilide of the formula I according to claim 1 or an agriculturally useful salt thereof.

17. A method for controlling harmful fungi, which method comprises treating the fungi, their habitat or the plants, areas, materials or spaces to be kept free from them with a fungicidally effective amount of at least one (hetero)cyclyl(thio)carboxanilide of the formula I according to claim 1 or an agriculturally useful salt thereof.

18. The (hetero)cyclylcarboxanilide of the formula I according to claim 2 in which A is a radical of the formula A-1a, A-2a or A-3a 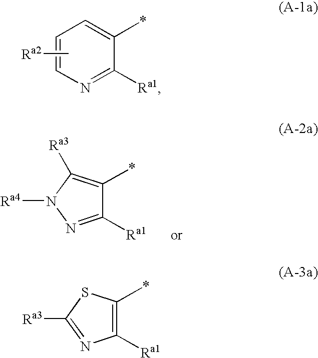
in which R^{a1}, R^{a2}, R^{a3} and R^{a4} have the meanings given in claim 2.

19. The (hetero)cyclylcarboxanilide of the formula I according to claim 2 in which R¹ is hydrogen.

20. The (hetero)cyclylcarboxanilide of the formula I according to claim 3 in which R¹ is hydrogen.
